SEQUENCE LISTING (5 sequences)

SEQ ID NO: 1 - DNA, 1257 bp, Homo sapiens
  source 1..1257: mol_type = genomic DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 2 - DNA, 1182 bp, synthetic construct
  source 1..1182: mol_type = other DNA
gaagatccacaaggtgatgctgctcagaaaacagacacctcacaccatgatcaagatcatccgacatttaacaagatcacacctaaccttgcagagttcgccttttccttgtatcgtcagcttgctcatcaaagcaactcgacgaacattttcttttccccagtaagtattgcaactgcatttgctatgctatcgttgggtaccaaagctgacactcatgacgaaatattggagggtctaaactttaacttgacagaaatccccgaagcccaaattcatgagggatttcaagagttgttgagaactctaaaccaacctgactctcaactgcagttaactaccggtaatggactgttcttaagcgaaggtttaaaattggtcgataagttccttgaggacgttaagaagttgtatcactctgaggcttttacggtcaatttcggagatactgaggaagccaagaaacaaatcaatgactacgtggaaaagggaactcaaggcaagatcgttgacttggtgaaagaactggatagagataccgtatttgctttagtgaactacatcttctttaaagggaaatgggaaagaccattcgaggtcaaggatactgaggaggaagattttcacgtcgaccaggtaaccactgttaaggttccgatgatgaaacgattgggaatgttcaacattcagcactgtaagaagttgtcaagttgggttctgcttatgaagtacttagggaatgcaactgccattttcttcttgcctgatgaaggtaaactgcaacatttggaaaatgaacttacacacgatattatcaccaaatttctagagaacgaagataggagatcagcctctttgcatttgccaaagctgtcaataacaggtacttatgacttgaaatccgttcttggccaattgggcataactaaggtgttttctaatggtgctgatttgagtggtgttacagaggaagctcccttaaagctatctaaggctgttcacaaagcagttcttaccattgacgagaaaggtactgaagctgcaggagctatgtttctggaagccattcctatgtccattccacctgaagttaaattcaataagccttttgtctttcttatgattgagcagaatacgaaatctccattattcatgggaaaggtagtgaatccaacccagaaa

SEQ ID NO: 3 - protein, 395 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDATEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - DNA, 1188 bp, synthetic construct
  source 1..1188: mol_type = other DNA
gaggaccctcaaggtgacgccgctcaaaagactgatacctcgcaccacgaccaagaccacccaacttttaacaagattactcctaatctagctgagttcgcattctctctctacagacaactcgctcatcagtctaactctacgaacattttcttctccccagtgtccattgcaactgctttcgccatgctttctttgggtactaaggctgacacacacgacgagattttagaaggattgaactttaatcttaccgaaattcctgaggcccaaattcacgaaggttttcaggagctgcttagaactcttaaccagccagatagccagctacaacttactactggaaatggactattcttgagtgaaggtctgaagctagttgacaaattcctcgaagatgtgaaaaagttgtaccattctgaggctttcaccgtcaacttcggtgacacagaggaggctaaaaagcagatcaacgactacgttgaaaagggtacgcaaggtaaaatcgttgacctcgttaaagagcttgatagagacaccgtatttgctcttgtcaattacatcttctttaaaggaaagtgggagagacctttcgaggtcaaggacaccgaggaggaagattttcatgtagatcaagtcaccactgttaaggttccaatgatgaagcgtctcggtatgtttaacatccaacactgtaaaaagctatcttcttgggtccttctgatgaagtacctgggtaacgctactgcaatcttcttcctaccagatgaaggtaagctccagcacctagagaacgaacttacccacgacatcattactaagttcctggaaaacgaagaccgtagatccgcttccctgcacctcccaaaactgtctatcactggtacctatgacctcaagtctgttctgggtcaactaggtataaccaaagtgttttcaaacggcgctgacctctccggtgtgactgaagaggcacctctcaagctgtccaaggccgttcacaaggcagtccttaccattgatgaaaagggaactgaagctgccggcgctatgttcctagaggccatcccaatgtcaattcctcccgaggttaagttcaacaagccattcgtcttcctaatgattgaacaaaacacaaagtctcctctgttcatgggaaaggttgttaacccaacccaaaagtaatag

SEQ ID NO: 5 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2025/0057927 A1
(43) Pub. Date: Feb. 20, 2025

(54) ALPHA-1 ANTITRYPSIN PRODUCED FROM YEAST FOR USE IN THE TREATMENT OF VIRAL INFECTIONS

(71) Applicant: AATec Medical GmbH, München (DE)

(72) Inventor: Manfred Stangl, Sauerlach (DE)

(21) Appl. No.: 18/723,224

(22) PCT Filed: Dec. 22, 2022
(86) PCT No.: PCT/EP2022/087461

§ 371 Date: Jun. 21, 2024

(30) Foreign Application Priority Data
Dec. 22, 2021 (EP) 21216836.3
Apr. 4, 2022 (EP) 22166483.2

(51) Int. Cl. A61K 38/55; A61K 9/00; A61P 31/14; C07K 14/81; C12N 1/16; C12N 15/81; C12R 1/84

(52) U.S. Cl. CPC A61K 38/55; A61K 9/0078; A61P 31/14; C07K 14/8125; C12N 1/16; C12N 15/815; C12R 2001/84

(57) ABSTRACT

A recombinant alphal-antitrysin (AAT) protein or a fragment thereof expressed in a genetically modified yeast for use in the treatment of a viral infection and/or lung inflammation. In one embodiment, the viral infection is a viral respiratory infection, preferably a coronavirus infection, such as a SARS CoV infection, more preferably SARS-CoV-2. Also disclosed is a method of producing recombinant AAT protein or fragment(s) thereof from genetically modified yeast. In embodiments, the method includes the steps of culturing a genetically modified yeast comprising an exogenous nucleic acid molecule with an AAT-encoding region operably linked to a promoter or promoter/enhancer combination, expressing recombinant AAT in the cultured yeast, and isolating recombinant AAT from the culture. In embodiments, the recombinant AAT produced from yeast is prepared in a pharmaceutical composition, such as a solution, preferably suitable for nebulization and subsequent inhalation by a subject.

The present invention is in the field of recombinant protein production, and treatment of viral infection and/or medical conditions associated with lung inflammation with said recombinant protein.

One aspect of the invention relates to a recombinant alpha1-antitrysin (AAT) protein or fragment thereof expressed in a genetically modified yeast for use in the treatment of a viral respiratory infection. In embodiments, the viral infection is a coronavirus infection, such as a SARS CoV infection, more preferably SARS-CoV-2.

A further aspect of the invention relates to a pharmaceutical composition comprising recombinant said AAT protein or fragment thereof and a pharmaceutically acceptable carrier. In embodiments, the pharmaceutical composition is suitable for inhalation by a subject. In embodiments, the composition is a solution suitable for inhalation, a soluble powder suitable for inhalation or a dry powder suitable for inhalation.

A further aspect of the invention relates to a method of producing recombinant AAT protein or fragment(s) thereof from genetically modified yeast. In embodiments, the method comprises the steps of culturing a genetically modified yeast comprising an exogenous nucleic acid molecule with an AAT-encoding region operably linked to a promoter or promoter/enhancer combination, expressing recombinant AAT in the cultured yeast, and isolating recombinant AAT from the culture. In embodiments, the recombinant AAT protein or fragment thereof is expressed in a yeast of the family Saccharomycesceae, preferably Saccharomycesceae Pichia, more preferably Pichia pastoris.

A further aspect of the invention relates to a method of producing a pharmaceutical composition, comprising the steps of producing recombinant AAT or fragment(s) thereof from a genetically modified yeast, and adding one or more pharmaceutically acceptable carriers to the recombinant AAT protein or fragment(s) thereof.

### BACKGROUND OF THE INVENTION

Severe acute respiratory syndrome (SARS) is a Coronavirus (CoV) mediated respiratory disease which was first observed in 2002. Based on scientific reports it is assumed that all human CoVs may be of zoonotic origin. Once a human becomes infected, the virus can quickly spread via droplet transmission and close contact between humans, leading to epidemic scenarios or even to a pandemic. As one example, “Coronavirus Disease 2019” (COVID-19) is caused by the pathogenic corona virus SARS-CoV-2. Beginning in December 2019, the virus spread globally within a few weeks and led to an international health emergency. The worldwide pandemic poses huge challenges to health systems and leads additionally to restrictions in social life and weakening of global market economies. Since no proven effective therapy is available to date, and the disease is associated with high morbidity and mortality, there is a great need for therapeutic interventions for those who are ill as well as for prophylactic measures to contain outbreaks.

Alpha-1 antitrypsin (also known as A1AT, AAT, PI, SERPINA1) is a ˜52 kDa glycoprotein that is one of the most abundant endogenous serine protease inhibitors (SERPIN superfamily). AAT is considered to be an acute phase protein, whereby AAT concentration can increase many fold upon acute inflammation.

Although known primarily for its anti-protease and anti-inflammatory activities, studies conducted over the past decade have cumulatively demonstrated that AAT is also an immune-modulator and a cytoprotective molecule. As such, microenvironments enriched with AAT were shown to contain reduced levels of pro-inflammatory cytokines, such as IL-1, IL-6, and TNF-α, and increased levels of anti-inflammatory mediators such as IL-1 receptor antagonist and IL-10. This phenomenon was also depicted in human PBMC in vitro studies, as well as in samples obtained from cystic fibrosis patients who received inhaled AAT. Concomitantly, AAT has been demonstrated to directly bind IL-8, and danger-associated molecular pattern molecules (DAMPs), such as extracellular HSP70 and gp96, which otherwise act as adjuvants to the associated immune responses (Lior et al., Expert Opinion on Therapeutic Patents, 2016).

Surprisingly, the anti-inflammatory qualities of AAT still allow innate immune cells to respond to authentic threats; macrophages readily phagocytose bacteria, neutrophils decontaminate infected sites, and antigen-loaded dendritic cells migrate to draining lymph nodes. T lymphocytes, on the other hand, respond to an AAT-rich environment indirectly, pending stimulation from innate immunocytes. For example, AAT has been shown to elicit semi-mature antigen presenting cells that favor the expansion of protective regulatory T cells. B lymphocytes, which belong to both the innate and adaptive immune system, appear to exert a modified response in the presence of AAT, in the form of diminished isotype switching, which results in enhanced protective IgM production. It has been suggested that the reduction in bacterial burden, as depicted in CF patients for instance, during treatment with AAT may relate to enhanced anti-pathogen immune responses; at the same time, local tissues are spared from inappropriate excessive injury that might promote deleterious adaptive responses by elevating the levels of local DAMPs (Lior et al., Expert Opinion on Therapeutic Patents, 2016).

Reports have indicated that the production of AAT is increased in COVID-19, but this anti-inflammatory response is overwhelmed in severe illness (McElvaney et al, Am J Respir Crit Care Med. 2020 Sep. 15; 202 (6): 812-821). The entry of SARS-CoV-2 into an infected cell is mediated by binding of the viral spike protein via its receptor binding domain (RBD) to the human angiotensin converting enzyme-2 (ACE2) target receptor. For example, blocking this interaction by human antibodies leads to a neutralization of the virus in patients and thus to a healing of the infection. Further work has also shown that entry of SARS-CoV-2 is facilitated by endogenous and exogenous proteases. These proteases proteolytically activate the SARS-CoV-2 spike glycoprotein and are key modulators of virus tropism. AAT has been identified as an abundant serum protease inhibitor that potently restricts protease-mediated entry of SARS-CoV-2. AAT inhibition of protease-mediated SARS-CoV-2 entry in vitro occurs at concentrations below what is present in serum and bronchoalveolar tissues, suggesting that AAT effects are physiologically relevant (Oguntuyo et al, bioRxiv. August 15; 2020).

However, to date, obtaining AAT still largely relies on purification from human plasma. For example, the available methods are essentially restricted to protein precipitation by addition of ammonium sulfate. The logical evolution in AAT purification was the combination of the ammonium sulfate fractionation with other procedures that could take advantage of the physico-chemical properties of AAT. To make easier the separation of the large pool of proteins (typically the result of ammonium sulfate precipitation) into several smaller pools, one (or more) of which was enriched in AAT, the first parameter considered by investigators was protein charge. More recently, an increased purification level was observed with the advent of a wide range of sophisticated materials in the ion-exchange and affinity chromatography field. For example, in Morihara et al, after saturating human plasma with ammonium sulfate (80%), the pellet was dissolved in phosphate buffer pH 8.0, dialyzed, and loaded on an Affi-GEL Blue column. Two subsequent steps on a Zn-chelate column followed by a DE-ion exchange chromatography allowed producing homogeneous AAT.

Kwon et al. were forerunners in the purification of recombinant AAT from yeast that was secreted in the medium as a glycosylated form. They developed a procedure that involved the precipitation of the protein with ammonium sulfate (60-75% saturation) followed by a series of subsequent chromatographic steps consisting of anion exchange (DEAE and mono Q columns) and a affinity (A-Gel Blue column) chromatography. Although the yeast-produced AAT was fully functional as a protease inhibitor (compared with the plasma form), the molecular mass of this protein (unlike plasma AAT), once treated with endoglycosidase H, decreased to that of recombinant AAT produced in Escherichia coli. This indicated that the N-linked glycosylation of this form was a high mannose-type. The authors also observed that glycosylation conferred the yeast-produced AAT with an enhanced kinetic stability toward heat inactivation. However, Saccharomyces diastaticus is not ideal for secretory production of recombinant proteins (Purification and characterization of alpha 1-antitrypsin secreted by recombinant yeast Saccharomyces diastaticus. J. Biotechnol. 1995, 42, 191-195).

Arjmand et al. 2011 (Avicenna J Med Biotechnol. 2011, 3 (3): 127-134) discloses the expression and purification of recombinant human AAT in a methylotrophic Yeast Pichia pastoris. Human AAT was expressed in a secretary manner and under the control of inducible alcohol oxidase 1 (AOX1) promoter. The amount of AAT protein in medium was measured as 60 mg/L, 72 hours after induction with methanol.

Arjmand et al. 2013 (Electronic Journal of Biotechnology, 2013, vol. 16, no. 1, 1-14) discloses the use of P. pastoris as a host for efficient production and secretion of recombinant AAT. The findings revealed that optimizing codon usage of the AAT gene for P. pastoris had positive effects on the level of secreted AAT under the control of inducible alcohol oxidase 1 (AOX1) and constitutive glycerol aldehyde phosphate dehydrogenase (GAP) promoters.

WO2021191900 discloses a method of treating COVID-19 with a pharmaceutical composition comprising recombinant human AAT in a dosage form suitable for inhalation.

There are still many drawbacks to address regarding AAT isolation from human plasma, for example the abundance of albumin in human plasma is challenging, and the yield of AAT from human plasma is limited, thus optimized methods for producing secreted recombinant proteins are needed. Furthermore, recombinant production of AAT in yeast has until now been limited by relatively low yields and clinical applications are lacking.

Despite advances in purification of AAT from human plasma, and advances in recombinant production of AAT, to the knowledge of the inventors there has been no convincing solution to date to address the disadvantages of the prior art. Methods and means are required that reduce or avoid the problems associated with isolating AAT from human plasma, and novel means and uses of recombinant AAT are required. Research in this field seeks new means to obtain a desired amount and purity level of AAT suitable for therapeutic use. Another objective problem which needs to be addressed is to provide novel treatments of viral respiratory disease and/or medical conditions associated with lung inflammation.

### SUMMARY OF THE INVENTION

In light of the prior art the technical problem underlying the present invention is to provide alternative and/or improved means for the treatment and/or prevention of viral infection and/or medical conditions associated with lung inflammation. Another problem underlying the invention is the provision of improved or alternative means for producing sufficient quantities and quality of AAT for therapeutic use. Another problem underlying the invention was the provision of novel means to treat coronavirus infections, or medical conditions associated with said infections.

These problems are solved by the features of the independent claims. Preferred embodiments of the present invention are provided by the dependent claims.

In one aspect, the invention relates to a recombinant alpha1-antitrysin (AAT) protein or a fragment thereof expressed in a genetically modified yeast for use in the treatment of a viral infection and/or lung inflammation.

In one embodiment, the invention relates to a recombinant alpha1-antitrysin (AAT) protein or a fragment thereof expressed in a genetically modified yeast for use in the treatment of a viral respiratory infection.

In embodiments, the viral infection or viral respiratory infection is a coronavirus infection, such as a SARS CoV infection, more preferably SARS-CoV-2.

As demonstrated in the examples below, the inventors have surprisingly found that recombinant AAT expressed in a genetically modified yeast is well suited for the treatment of viral respiratory infections, in particular SARS CoV, such as SARS-CoV-2.

Although AAT production in yeast has been described previously, to the knowledge of the inventors the efficacy of recombinant AAT from yeast in treating a viral respiratory disease has not been previously assessed. Surprisingly, experimental work presented in the examples below demonstrates that not only is recombinant AAT from yeast capable of blocking viral entry into model cells in vitro, but improvements are observed over the existing therapeutic formulations of purified AAT from human plasma. For example, recombinant isolated AAT from yeast shows comparatively superior properties in blocking viral pseudoparticle entry in Caco2 target cells compared to the Prolastin AAT preparation.

The present invention therefore represents a novel and surprisingly effective recombinant AAT preparation using yeast as an expression system. Correspondingly, the isolated recombinant AAT protein itself, the genetically modified yeast used to produce the recombinant AAT and methods of preparing and using the AAT produced in yeast may, in embodiments, be defined by the inventive finding of efficacy in treating a viral respiratory disease.

### Embodiments Regarding Medical Use and Medical Indications:

In one aspect, the invention relates to a recombinant alpha1-antitrysin (AAT) protein or a fragment thereof expressed in a genetically modified yeast for use in the treatment of a viral respiratory infection.

In embodiments, the invention relates to recombinant alpha1-antitrysin (AAT) protein or a fragment thereof expressed in a genetically modified yeast for use in the treatment of a medical condition associated with a viral respiratory infection.

In one embodiment, the viral respiratory infection is a coronavirus infection, preferably a SARS coronavirus infection, more preferably SARS-CoV-2.

In embodiments, the invention relates to a recombinant alpha1-antitrysin (AAT) protein or a fragment thereof expressed in a genetically modified Pichia pastoris yeast strain for use in the treatment of a viral respiratory infection.

Despite AAT being known to be effective in the treatment of lung disease, the use of AAT expressed in yeast represents an unexpectedly useful and effective means to treating viral infections of the lung or respiratory system. Firstly, lung diseases as such vary greatly with respect to their pathological causes, and viral lung disease is mechanistically and biologically distinct from other lung diseases. The effectiveness of treating viral diseases of the lung as in the present invention could not have been expected per se from earlier disclosures regarding AAT treatment of other lung diseases, for example those based on non-viral inflammation or chronic conditions, such as immune diseases or chronic obstructive pulmonary disease (COPD).

In the context of the present invention, the use of a recombinant alpha 1-antitrysin (AAT) protein or a fragment thereof expressed in a genetically modified yeast is not limited to treating a SARS-CoV infection, such as SARS-CoV-2. The viral infection to be treated may, in embodiments, also refer to other human coronavirus infections, such as human corona viruses NL63, 229E, HKU1 and/or OC43 or coronaviruses derived therefrom.

In embodiments, the condition to be treated is a form of COVID-19, which may be asymptomatic, mild, moderate or severe.

In embodiments, the recombinant AAT as described herein is for use in the prevention and/or treatment of Long COVID or a medical condition associated with Long COVID.

In embodiments, the patient is subjected additionally to a standard medical treatment for COVID-19. For example, standard medical treatments comprise oxygen support, non-invasive ventilation, high flow oxygen, mechanical ventilation and extracorporeal membrane oxygenation.

In embodiments, the patient is subjected additionally to a standard medical treatment for COVID-19 and/or for other viral infections, such as influenza and RSV. For example, one or more painkillers, anti-fever preparations and/or anti-inflammatoires may be administered in combination with the AAT treatment.

In embodiments, the patient to be treated has COVID-19 symptoms, which may be present of any duration or intensity, for example the patient may exhibit acute, delayed and/or ongoing COVID symptoms.

In embodiments of the invention, the recombinant AAT is administered for a plurality of consecutive days. In embodiments, the patient may receive treatment via inhalation multiple times within one day, multiple times within one week, or even on an ongoing basis over multiple weeks.

In embodiments of the invention, the recombinant AAT is administered as preventative treatment, for example to achieve short-term prevention of viral infection in a respiratory tract by administration of the AAT shortly before exposure or potential exposure to a virus.

In embodiments of the invention, the treatment results in at least one outcome of enhanced viral clearance, reduced hospitalization, reduced oxygen dependence, reduced intensive care or mechanical ventilation need, reduced healthcare utilization or burden, reduced absences from school or work, decreased antibiotic need, decreased steroid need, decreased relapse frequency and/or decreased morbidity or risk of morbidity.

In embodiments, the time of a subject exhibiting symptoms of a viral respiratory disease, such as influenza or COVID, is reduced using the treatment of the invention. The symptoms of the medical condition may be reduced to an extent greater than if the AAT has not been administered. Symptoms may therefore be reduced to less than two weeks, less than 10 days, less than 7 days, or less than 6, 5, 4, 3, 2 or less than 1 day of symptoms of a viral respiratory disease.

In embodiments, the treatment with AAT comprises additionally at least one other therapeutic agent. Such an agent may be, without limitation, an antiviral agent, for example an antiviral agent selected from the group consisting of a protease inhibitor, a helicase inhibitor, a viral replication inhibitor, and a virus cell entry inhibitor.

In embodiments, the patient is subjected additionally to an anti-viral treatment, selected from, without limitation, neuraminidase inhibitors (NAIs), such as Tamiflu (oseltamivir), Alpivab (peramivir), Relenza (zanamivir), or Zanamivir (intravenous formulation), or one or more M2 inhibitors (adamantanes), such as amantadine or rimantadine.

In embodiments, the invention relates to a recombinant alpha1-antitrysin (AAT) protein or a fragment thereof expressed in a genetically modified yeast for use in the treatment or prevention of any given viral infection of the respiratory tract.

By way of examples, respiratory virus disease may occur from any infection by an adenovirus, coronaviruses (common cold viruses), influenza (flu), parainfluenza, parvovirus b19 (fifth disease), respiratory syncytial virus (RSV) or rhinovirus (common cold). Further viral respiratory disease or virus may relate to HBoV, human bocavirus; HCoV, human coronavirus; HMPV, human metapneumovirus; HPIV, human parainfluenza virus; HRSV, human respiratory syncytial virus; HRV, human rhinovirus; PCF, pharyngoconjunctival fever; SARS, severe acute respiratory syndrome; SARS-CoV, Coronavirus associated with SARS; URI, upper respiratory infection.

In embodiments, the invention also relates to a recombinant alpha1-antitrysin (AAT) protein or a fragment thereof expressed in a genetically modified yeast for use in the treatment or prevention of an influenza infection.

In embodiments, the invention also relates to a recombinant alpha1-antitrysin (AAT) protein or a fragment thereof expressed in a genetically modified yeast for use in the treatment or prevention of a viral respiratory disease, preferably one that is susceptible to protease inhibition as achieved by AAT, as shown in the examples below.

In one embodiment, the recombinant AAT protein is administered to treat a viral infection SARS-CoV-2, influenza or RSV, wherein the AAT protein or fragment thereof is expressed from Pichia pastoris and the AAT protein or fragment thereof is administered by inhalation of a nebulized solution.

In one embodiment, the recombinant AAT protein is administered to treat a viral infection SARS-CoV-2, and the AAT protein or fragment thereof is expressed from Pichia pastoris and isolated using chromatography, such as ion exchange or hydrophobic interaction chromatography, and the AAT protein or fragment thereof is administered by inhalation of a nebulized solution.

### Embodiments Regarding AAT Sequences:

In one embodiment, the AAT protein or fragment thereof comprises a sequence according to SEQ ID NO 3 or 5, or is encoded by SEQ ID NO 1, 2 or 4.

In the context of the present invention, SEQ ID NO 1, 3 and 5 are exemplary nucleotide sequences encoding AAT-protein. The nucleotide sequence encoding AAT according to the invention is not limited to SEQ ID NO 1, 3 and 5 and may, in embodiments, be optimized for expression, for example a codon optimized nucleotide sequence encoding AAT-protein.

In embodiments, the AAT protein or fragment thereof comprises or consists of the sequence according to SEQ ID NO 3 or 5. These sequences are exemplary sequences and non-limiting to the present invention.

Sequence variations, comprising for example amino acid sequence changes via substitution and/or deletion, and/or in length, are disclosed in more detail below.

### Embodiments Regarding AAT Produced in Yeast:

As described in more detail herein, the recombinant AAT of the present invention is expressed from a yeast. Despite therapeutic usefulness of isolated human AAT (from plasma) being known to a skilled person, the activity of yeast AAT protein in the specific context of treating viral respiratory diseases could not have been expected. The yeast origin of the recombinant AAT as described herein enables not only greater amounts of AAT to be produced, due to recombinant expression systems in yeast, but the yeast-expressed AAT also achieves unexpected therapeutic effects in the context of treating viral infections. As shown in the examples below, recombinant AAT from yeast appears to achieve a greater effect on blocking viral entry into target cells in vitro, compared to AAT isolated from plasma.

Without being bound to preferred and non-limiting examples, the yeast strains and means of expression as described herein may, in embodiments, play a role in obtaining the beneficial properties of the recombinant yeast described herein. The source of the recombinant AAT, with respect to yeast strain, expression vectors, use of particular promoters or culture conditions may provide inherent physical and/or functional characteristics to the therapeutic recombinant AAT protein itself. Features related to the expression or manufacture of the recombinant AAT from yeast may therefore, in embodiments, be used to define the recombinant AAT protein of the invention.

In one embodiment, the recombinant AAT protein or fragment thereof is expressed in a yeast of the family Saccharomycesceae, preferably Saccharomycesceae Pichia, more preferably Pichia pastoris.

In one embodiment, the recombinant AAT or fragment thereof has a serum half-life and/or activity not less than AAT purified from human plasma.

In one embodiment, the AAT or fragment thereof comprises post-translational modifications of the recombinant AAT protein or fragment thereof.

In one embodiment, the post-translation modification is O-glycosylation, N-glycosylation, N-terminal methionine removal, N-acetylation and/or phosphorylation or any combination thereof, preferably comprising one or more specific modifications from expression in yeast, such as Man3GlcNAc2.

In one embodiment, the recombinant AAT protein or fragment thereof comprises one or more human-like glycoform patterns.

In one embodiment, the AAT protein or fragment thereof is produced from genetically modified yeast, in a method comprising the steps of:
a. culturing a genetically modified yeast comprising an exogenous nucleic acid molecule with an AAT-encoding region operably linked to a promoter or promoter/enhancer combination,
b. expressing recombinant AAT in the cultured yeast, and
c. isolating recombinant AAT from the culture.

In one embodiment, the AAT protein or fragment thereof is produced from genetically modified yeast, in a method comprising the steps of:
a culturing a genetically modified yeast comprising an exogenous nucleic acid molecule with an AAT-encoding region operably linked to a promoter or promoter/enhancer combination, wherein said yeast is preferably of the family Saccharomycesceae, preferably Saccharomycesceae Pichia, more preferably Pichia pastoris,
b. expressing recombinant AAT or fragment thereof in the cultured yeast, wherein the promoter is preferably glyceraldehyde-3-phosphate dehydrogenase (GAP) or alcohol oxidase I (AOX1), and
c. isolating recombinant AAT or fragment thereof from the culture.
   Embodiments Regarding a Method of Producing AAT from Yeast:

In another aspect, the invention relates to a method of producing recombinant alpha1-antitrysin (AAT) protein or fragment(s) thereof from genetically modified yeast, comprising the steps of:
a. culturing a genetically modified yeast comprising an exogenous nucleic acid molecule with an AAT-encoding region operably linked to a promoter or promoter/enhancer combination,
b. expressing recombinant AAT in the cultured yeast, and
c. isolating recombinant AAT from the culture.

As described herein, the recombinant AAT obtained by the method for producing AAT, as described herein, is preferably configured for medical use in treating viral infections, preferably viral respiratory infections.

In embodiments, the yeast as used herein can be any yeast species suitable for expressing recombinant protein.

In embodiments, the yeast is one or more of a Saccharomyces, Pichia, Hansenula, Yarrowia, Arxula, Kluyveromyces, or Schizosaccharomyces species, for example Saccharomyces cerevisiae, Pichia pastoris, Hansenula polymorpha, Yarrowia lipolytica, Arxula adeninivorans, Kluyveromyces lactis or Schizosaccharomyces pombe.

In embodiments, the yeast is of the family Saccharomycesceae, preferably Saccharomycesceae Pichia.

In embodiments, the yeast is Pichia pastoris.

In embodiments, the yeast is Saccharomyces cerevisiae.

In embodiments, the yeast is Schizosaccharomyces pombe.

Without being bound by theory, using a yeast host to express the recombinant AAT (preferably rhAAT) confers a structure, function and/or activity to the AAT that is distinct from AAT isolated from a human source, such as human plasma. The definition of the AAT as being recombinantly expressed from yeast therefore represents a structural and/or functional feature to the AAT protein itself.

In embodiments, post-translational modifications of the AAT protein induced via expression in yeast allow a combination of excellent AAT activity and unusually high yields via expression and secretion to a culture supernatant.

In embodiments, expression in yeast enables one or more of increased stability, increased purity, increased activity and/or increased resistance to degradation post-administration, of the yeast AAT over enriched and/or isolated human AAT preparations.

In embodiments, the AAT produced in yeast exhibits surprisingly few unwanted side effects after administration in human subjects. The inventors envisage that AAT expressed from yeast shows good tolerability profiles in human subjects and a low risk of unwanted immune reactions to the AAT preparation.

In embodiments, the particular yeast strain employed for recombinant AAT production confers a structure, function and/or activity to the AAT that is distinct from AAT produced in other yeast strains and/or AAT isolated from a human source, such as human plasma. For example, the examples provided herein show unexpectedly good expression, secretion, and isolation of recombinant AAT from Pichia pastoris. In embodiments, the AAT expressed from Pichia pastoris exhibits one or more advantages with respect to structure, function, activity, expression, secretion, and/or isolation over expression of AAT in other yeast hosts and/or over isolated human AAT.

In one embodiment, the method as used herein comprises use of an inducible promoter configured to regulate the expression of AAT in yeast, such as alcohol oxidase I (AOX1).

In embodiments, the AAT protein or fragment(s) thereof is of an origin selected from the group consisting of mammalian, fish, fungi and plant, preferably wherein the AAT protein is a human AAT protein or fragment thereof.

In embodiments, the AAT protein or fragment(s) is secreted from the yeast into the medium, to a concentration of at least 1 mg protein per litre of liquid medium (mg/L), preferably to a concentration of 2 to 100 mg/L, more preferably to a concentration of 5 to 100 mg/mL, or 10 to 100 mg/L.

In embodiments, the AAT protein or fragment(s) thereof secreted from the yeast to the medium amounts to a concentration of 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 mg/L. The yield may also be in a range derived from any one or more of the values provided above. In embodiments, the AAT protein or fragment(s) thereof secreted from the yeast to the medium amounts to a concentration above the values presented herein.

In one embodiment, the genetically modified yeast is of the family Saccharomycesceae, preferably Saccharomycesaceae Saccharomyces, more preferably Saccharomyces cerevisiae, wherein said yeast produces also Xanthohumol. In embodiments, the genetically modified yeast is a Pichia strain, and produces also Xanthohumol.

The genetically modified yeast used to express the recombinant AAT preferably comprises a yeast expression vector, configured for effective AAT expression. Suitable vectors that comprise replication and expression components for a yeast system, are known to a skilled person. Without limitation, suitable vectors are those described herein, such as pGAPz, pPICz and/or pEX-A258.

As described herein, an exogenous nucleic acid molecule with an AAT-encoding region is employed, such as a yeast expression vector, wherein said AAT encoding region is operably linked to a promoter or promoter/enhancer combination. Suitable promoters can be selected by a skilled person.

In embodiments, the promoter is glyceraldehyde-3-phosphate dehydrogenase (GAP).

In other embodiments, the promoter is inducible, preferably alcohol oxidase I (AOX1).

In embodiments, expression of AAT from a genetically modified yeast typically leads to secretion of soluble AAT in the supernatant of the yeast culture. The recombinant AAT typically requires isolation, or purification, before further use. Clinical grade AAT can also be generated using the requisite protocols.

In embodiments, the recombinant AAT of the invention is isolated using ion-exchange and/or affinity and/or hydrophobic interaction chromatography and/or size exclusion chromatography. In embodiments, the yeast culture supernatant may be treated with ammonium sulfate to precipitate protein. Subsequently, the precipitated pellet is dissolved in a suitable buffer, such as a phosphate buffer at pH 8.0, optionally dialyzed, and loaded to an appropriate column for purification. For example, ion exchange chromatography allows production of high purity recombinant AAT.

In embodiments, purification of a supernatant of recombinant AAT produced in yeast comprises cation exchange chromatography (CEX).

In embodiments, purification of a supernatant of recombinant AAT produced in yeast comprises anion exchange chromatography (AEX).

In embodiments, purification of a supernatant of recombinant AAT produced in yeast comprises size exclusion chromatography.

In embodiments, purification of a supernatant of recombinant AAT produced in yeast comprises hydrophobic interaction chromatography.

In embodiments, purification of a supernatant of recombinant AAT produced in yeast comprises affinity chromatography.

In embodiments, the AAT may be expressed in yeast as a fusion protein to a suitable purification tag, such as a histidine tag (His-tag), which facilitates a later purification step.

In embodiments, the method of preparing AAT from yeast may comprise modifying the recombinant AAT protein in vitro after isolation from the yeast. In embodiments, the modification comprises covalent attachment of recombinant AAT protein to a biocompatible polymer.

### Embodiments Regarding a Composition and Administration Forms:

A further aspect of the invention relates to a pharmaceutical composition comprising said recombinant AAT protein or fragment thereof produced in yeast and a pharmaceutically acceptable carrier.

In embodiments, the pharmaceutical composition is suitable for inhalation by a subject. In embodiments, the composition is a solution suitable for inhalation, a soluble powder suitable for inhalation or a dry powder suitable for inhalation.

In embodiments, administration by inhalation may encompass one or more of inhaling recombinant AAT or fragments thereof, produced in yeast, via a solution suitable for inhalation, a soluble powder suitable for inhalation or a dry powder suitable for inhalation.

In one embodiment, the AAT protein or fragment thereof is administered by inhalation of a nebulized solution.

In embodiments, the pharmaceutical composition can be prepared by a nebulizer for inhalation, for example by a Pari Boy Pro, or similar device.

In one embodiment, the viral infection is a coronavirus infection, such as SARS CoV, preferably SARS-CoV-2, and the AAT protein or fragment thereof is administered by inhalation of a nebulized solution.

In a related aspect, the present invention relates to a pharmaceutical composition comprising recombinant alpha1-antitrysin (AAT) protein or a fragment thereof expressed in a genetically modified yeast for use in the treatment of a viral respiratory infection.

In one embodiment, the viral infection is SARS-CoV-2, the AAT protein or fragment thereof is expressed in Pichia pastoris, and the AAT is administered by inhalation of a nebulized solution.

In embodiments, compositions of the present invention are formulated for parenteral administration, e.g., intravascular (intravenous or intraarterial), intraperitoneal or intramuscular administration.

In embodiments, the pharmaceutical composition for use can be accompanied by simultaneous or sequential administration of one or more other active agents useful for treating a viral infection or a medical condition associated with lung inflammation.

In one embodiment, the one or more other active agents can be Xanthohumol.

Without being bound by theory, during the fermentation process using yeast of the family Saccharomycesceae, preferably Saccharomycesceae Saccharomyces, more preferably Saccharomyces cerevisiae, when hops is added to the process, Xanthohumol is produced as a by-product and functions as a potent pan-inhibitor for various coronaviruses by targeting a protease of the coronaviruses, for example, SARS-CoV-2.

Wang et al., 2021 shows Xanthohumol inhibited protease activities in the enzymatical assays, while pretreatment with Xanthohumol restricted the SARS-CoV-2 and PEDV replication in Vero-E6 cells. Xanthohumol is a potent pan-inhibitor of coronaviruses and an excellent lead compound for further drug development (Wang et al., 2021, Xanthohumol Is a Potent Pan-Inhibitor of Coronaviruses Targeting Main Protease, international journal of molecular sciences).

Therefore, in embodiments, a genetically modified yeast from family of Saccharomycesaceae, preferably Saccharomycesceae Saccharomyces, more preferably Saccharomyces cerevisiae produces not only recombinant AAT protein, but also an auxiliary substance Xanthohumol which may lead to a synergistic effect for treating viral infections, such as coronaviruses.

In embodiments, the invention relates to a method for the prevention, reduction of risk and/or treatment of a viral respiratory infection, the method comprising administering a therapeutically amount of recombinant AAT protein, wherein said recombinant AAT protein or fragment thereof is expressed in a genetically modified yeast as described herein, to a subject in need thereof, wherein said administering comprises preferably inhalation of AAT protein, for example of nebulized droplets, and/or injection of AAT protein to the subject.

### Further Aspects of the Invention:

Another aspect of present invention relates to a recombinant AAT protein or fragment thereof obtainable from the method as described in any embodiment of the invention.

A further aspect of present invention relates to a genetically modified yeast, wherein said yeast is genetically modified to express recombinant AAT and fragment(s) thereof, comprising an exogenous nucleic acid molecule with an AAT encoding region operably linked to a promoter or promoter/enhancer combination.

A further aspect of present invention relates to a recombinant AAT protein or a fragment thereof produced by expression in yeast.

A further aspect of present invention relates to a recombinant AAT protein or fragment thereof produced from steps comprising
a. culturing a genetically modified yeast comprising an exogenous nucleic acid molecule with an AAT-encoding region operably linked to a promoter or promoter/enhancer combination, such as glyceraldehyde-3-phosphate dehydrogenase (GAP), alcohol oxidase I (AOX1)
b. expressing recombinant AAT in the cultured yeast, and
c. isolating recombinant AAT from the culture.

A further aspect of the present invention relates to a method of producing a pharmaceutical composition, comprising the steps of
a. producing recombinant AAT or fragment(s) thereof from a genetically modified yeast according to a method as described herein, and
b. adding one or more pharmaceutically acceptable carriers to the recombinant AAT protein or fragment(s) thereof.

Further exemplary aspects and embodiments of the invention relate, for example, to:

Embodiment 1.A method of producing recombinant alpha1-antitrysin (AAT) protein or fragment(s) thereof from genetically modified yeast, comprising the steps of:
a culturing a genetically modified yeast comprising an exogenous nucleic acid molecule with an AAT-encoding region operably linked to a promoter or promoter/enhancer combination,
b. expressing recombinant AAT in the cultured yeast, and
c. isolating recombinant AAT from the culture.

Embodiment 2. The method according to the preceding embodiment, wherein the promoter is an inducible promoter configured to regulate the expression of AAT in yeast, such as alcohol oxidase I (AOX1).

Embodiment 3. The method according to any one of the preceding embodiments, wherein the yeast is of the family Saccharomycesaceae, preferably Saccharomycesceae Pichia, more preferably Pichia pastoris.

Embodiment 4. The method according to any one of the preceding embodiments, wherein the AAT protein or fragment(s) thereof is of an origin selected from the group consisting of mammalian, fish, fungi and plant, preferably wherein the AAT protein is a human AAT protein.

Embodiment 5. The method according to any one of the preceding embodiments, wherein the exogenous nucleic acid molecule comprises a sequence as set forth in SEQ ID NO: 1 or SEQ ID NO: 2.

Embodiment 6. The method according to any one of the preceding embodiments, wherein the recombinant AAT has a serum half-life and/or activity not less than AAT purified from human plasma.

Embodiment 7. The method according to any one of the preceding embodiments, wherein the AAT protein or fragment(s) is secreted from the yeast into the medium, to a concentration of at least 1 mg protein per litre of liquid medium (mg/L), preferably to a concentration of 5-100 mg/L.

Embodiment 8. The method according to any one of the preceding embodiments, comprising post-translational modifications of the recombinant AAT protein.

Embodiment 9. The method according to any one of the preceding embodiments, wherein the post-translation modification is O-glycosylation, N-glycosylation, N-terminal methionine removal, N-acetylation and/or phosphorylation or any combination thereof.

Embodiment 10. The method according to any one of the preceding embodiments, wherein the recombinant AAT protein has one or more human-like glycoform patterns.

Embodiment 11. The method according to any one of the preceding embodiments, comprising modifying the recombinant AAT protein in vitro after isolation from the yeast.

Embodiment 12. The method according to the preceding embodiment, wherein the modification is covalent attachment of recombinant AAT protein to a biocompatible polymer.

Embodiment 13. A recombinant AAT protein or fragment thereof obtainable from a method according to any one of the preceding embodiments.

Embodiment 14. The recombinant AAT protein or fragment thereof according to the preceding embodiment, comprising one or more specific modifications from expression in yeast, such as Man3GlcNAc2.

Embodiment 15. A genetically modified yeast, wherein said yeast is genetically modified to express recombinant AAT and fragment(s) thereof, comprising an exogenous nucleic acid molecule with an AAT encoding region operably linked to a promoter or promoter/enhancer combination.

Embodiment 16. The genetically modified yeast according to the preceding embodiment, wherein the exogenous nucleic acid molecule comprises a sequence as set forth in SEQ ID NO: 1 or SEQ ID NO: 2.

Embodiment 17. The genetically modified yeast according to any one of embodiments 15 and 16, wherein the promoter is glyceraldehyde-3-phosphate dehydrogenase (GAP).

Embodiment 18. The genetically modified yeast according to any one of embodiments 15-17, wherein the promoter is inducible, preferably alcohol oxidase I (AOX1).

Embodiment 19. The genetically modified yeast according to any one of embodiments 15-18, wherein said yeast is of the family Saccharomycesaceae, preferably Saccharomycesaceae Pichia, more preferably Pichia pastoris.

Embodiment 20. A recombinant AAT protein or fragment thereof produced from steps comprising
a. culturing a genetically modified yeast comprising an exogenous nucleic acid molecule with an AAT-encoding region operably linked to a promoter or promoter/enhancer combination, such as glyceraldehyde-3-phosphate dehydrogenase (GAP), alcohol oxidase I (AOX1)
b. expressing recombinant AAT in the cultured yeast, and
c. isolating recombinant AAT from the culture, wherein said yeast is of the family Saccharomycesceae, preferably Saccharomycesceae Pichia, more preferably Pichia pastoris.

Embodiment 21. A recombinant AAT protein or fragment thereof according to the preceding embodiment, wherein the exogenous nucleic acid molecule comprises a sequence as set forth in SEQ ID NO: 1 or SEQ ID NO: 2, and/or wherein the AAT amino acid sequence comprises a sequence according to SEQ ID NO: 3 or SEQ ID NO: 5.

Embodiment 22. A recombinant AAT protein or fragment thereof according to embodiments 20-21, comprising post-translational modifications, for example, O-glycosylation, N-glycosylation, N-terminal methionine removal, N-acetylation and/or phosphorylation and any combination thereof.

Embodiment 23. A recombinant AAT protein or fragment thereof according to embodiments 20-22, comprising one or more human-like glycoform patterns.

Embodiment 24. A method of producing a pharmaceutical composition, comprising the steps of
a. producing recombinant AAT or fragment(s) thereof from a genetically modified yeast according to a method of any one of embodiments 1-12, and
b. adding one or more pharmaceutically acceptable carriers to the recombinant AAT protein or fragment(s) thereof.

Embodiment 25. A pharmaceutical composition comprising recombinant AAT protein or fragment thereof and a pharmaceutically acceptable carrier, wherein said recombinant AAT protein or fragment thereof is expressed in a genetically modified yeast according to any one of the embodiments 15-19.

Embodiment 26. The pharmaceutical composition according to the preceding embodiment, wherein the composition is a solution suitable for inhalation or soluble powder suitable for inhalation or dry powder direct for inhalation.

Embodiment 27. The pharmaceutical composition according to the preceding embodiment, wherein the composition can be prepared by a nebulizer for inhalation.

Embodiment 28. The pharmaceutical composition according to embodiment 25 for use in the prevention and/or treatment of a viral infection.

Embodiment 29. The pharmaceutical composition for use according to the preceding embodiment, wherein the viral infection is caused by a Coronavirus, preferably by SARS-CoV-2.

Embodiment 30. The pharmaceutical composition according to embodiment 25 for use in the prevention and/or treatment of medical condition associated with lung inflammation.

Embodiment 31. The pharmaceutical composition for use according to embodiments 28-30, further comprises simultaneous or sequential administration of one or more other active agents useful for treating a viral infection or medical condition associated with lung inflammation.

The various aspects and embodiments of the invention are unified by, benefit from, are based on and/or are linked by the common and surprising finding that a recombinant AAT protein produced from genetically engineered yeast may be effectively applied in treating a viral infection and/or medical conditions associated with lung inflammation. Any given feature describing one or more aspects or embodiments of the invention may be used to describe, and is considered disclosed in the context of, any other aspect or embodiment of the invention. For example, the embodiments of the invention relating to the method of AAT production may be considered as related to, and disclosed in the context of, the other aspects of the invention, such as the AAT protein as such and/or medical uses thereof.

Overview of sequences disclosed in the description:

| SEQ ID | | |
|---|---|---|
| NO | Sequence | Description |
| 1 | ATGCCGTCTTCTGTCTCGTGGGGCATCCTCCTGCTGGCAGGCCTG | AAT coding |
| | TGCTGCCTGGTCCCTGTCTCCCTGGCTGAGGATCCCCAGGGAGAT | sequence |
| | GCTGCCCAGAAGACAGATACATCCCACCATGATCAGGATCACCCAA | |
| | CCTTCAACAAGATCACCCCCAACCTGGCTGAGTTCGCCTTCAGCCT | |
| | ATACCGCCAGCTGGCACACCAGTCCAACAGCACCAATATCTTCTTC | |
| | TCCCCAGTGAGCATCGCTACAGCCTTTGCAATGCTCTCCCTGGGGA | |
| | CCAAGGCTGACACTCACGATGAAATCCTGGAGGGCCTGAATTTCAA | |
| | CCTCACGGAGATTCCGGAGGCTCAGATCCATGAAGGCTTCCAGGA | |
| | ACTCCTCCGTACCCTCAACCAGCCAGACAGCCAGCTCCAGCTGAC | |
| | CACCGGCAATGGCCTGTTCCTCAGCGAGGGCCTGAAGCTAGTGGA | |
| | TAAGTTTTTGGAGGATGTTAAAAAGTTGTACCACTCAGAAGCCTTCA | |
| | CTGTCAACTTCGGGGACACCGAAGAGGCCAAGAAACAGATCAACG | |
| | ATTACGTGGAGAAGGGTACTCAAGGGAAAATTGTGGATTTGGTCAA | |
| | GGAGCTTGACAGAGACACAGTTTTTGCTCTGGTGAATTACATCTTCT | |
| | TTAAAGGCAAATGGGAGAGACCCTTTGAAGTCAAGGACACCGAGG | |
| | AAGAGGACTTCCACGTGGACCAGGTGACCACCGTGAAGGTGCCTA | |
| | TGATGAAGCGTTTAGGCATGTTTAACATCCAGCACTGTAAGAAGCT | |
| | GTCCAGCTGGGTGCTGCTGATGAAATACCTGGGCAATGCCACCGC | |
| | CATCTTCTTCCTGCCTGATGAGGGGAAACTACAGCACCTGGAAAAT | |
| | GAACTCACCCACGATATCATCACCAAGTTCCTGGAAAATGAAGACA | |
| | GAAGGTCTGCCAGCTTACATTTACCCAAACTGTCCATTACTGGAAC | |
| | CTATGATCTGAAGAGCGTCCTGGGTCAACTGGGCATCACTAAGGTC | |
| | TTCAGCAATGGGGCTGACCTCTCCGGGGTCACAGAGGAGGCACCC | |
| | CTGAAGCTCTCCAAGGCCGTGCATAAGGCTGTGCTGACCATCGAC | |
| | GAGAAAGGGACTGAAGCTGCTGGGGCCATGTTTTTAGAGGCCATA | |
| | CCCATGTCTATCCCCCCCGAGGTCAAGTTCAACAAACCCTTTGTCT | |
| | TCTTAATGATTGAACAAAATACCAAGTCTCCCCTCTTCATGGGAAAA | |
| | GTGGTGAATCCCACCCAAAAATAA | |
| 2 | GAAGATCCACAAGGTGATGCTGCTCAGAAAACAGACACCTCACACC | Codon |
| | ATGATCAAGATCATCCGACATTTAACAAGATCACACCTAACCTTGCA | optimized |
| | GAGTTCGCCTTTTCCTTGTATCGTCAGCTTGCTCATCAAAGCAACTC | AAT coding |
| | GACGAACATTTTCTTTTCCCCAGTAAGTATTGCAACTGCATTTGCTA | sequence 1 |
| | TGCTATCGTTGGGTACCAAAGCTGACACTCATGACGAAATATTGGA | |
| | GGGTCTAAACTTTAACTTGACAGAAATCCCCGAAGCCCAAATTCAT | |
| | GAGGGATTTCAAGAGTTGTTGAGAACTCTAAACCAACCTGACTCTC | |
| | AACTGCAGTTAACTACCGGTAATGGACTGTTCTTAAGCGAAGGTTTA | |
| | AAATTGGTCGATAAGTTCCTTGAGGACGTTAAGAAGTTGTATCACTC | |
| | TGAGGCTTTTACGGTCAATTTCGGAGATACTGAGGAAGCCAAGAAA | |
| | CAAATCAATGACTACGTGGAAAAGGGAACTCAAGGCAAGATCGTTG | |
| | ACTTGGTGAAAGAACTGGATAGAGATACCGTATTTGCTTTAGTGAAC | |
| | TACATCTTCTTTAAAGGGAAATGGGAAAGACCATTCGAGGTCAAGG | |
| | ATACTGAGGAGGAAGATTTTCACGTCGACCAGGTAACCACTGTTAA | |
| | GGTTCCGATGATGAAACGATTGGGAATGTTCAACATTCAGCACTGT | |
| | AAGAAGTTGTCAAGTTGGGTTCTGCTTATGAAGTACTTAGGGAATG | |
| | CAACTGCCATTTTCTTCTTGCCTGATGAAGGTAAACTGCAACATTTG | |
| | GAAAATGAACTTACACACGATATTATCACCAAATTTCTAGAGAACGA | |
| | AGATAGGAGATCAGCCTCTTTGCATTTGCCAAAGCTGTCAATAACA | |
| | GGTACTTATGACTTGAAATCCGTTCTTGGCCAATTGGGCATAACTAA | |
| | GGTGTTTTCTAATGGTGCTGATTTGAGTGGTGTTACAGAGGAAGCT | |
| | CCCTTAAAGCTATCTAAGGCTGTTCACAAAGCAGTTCTTACCATTGA | |
| | CGAGAAAGGTACTGAAGCTGCAGGAGCTATGTTTCTGGAAGCCATT | |
| | CCTATGTCCATTCCACCTGAAGTTAAATTCAATAAGCCTTTTGTCTTT | |
| | CTTATGATTGAGCAGAATACGAAATCTCCATTATTCATGGGAAAGGT | |
| | AGTGAATCCAACCCAGAAA | |
| 3 | EDPQGDAAQKTDTSHHDQDHPTFNKITPNLAEFAFSLYRQLAHQSNST | Human |
| | NIFFSPVSIATAFAMLSLGTKADTHDEILEGLNFNLTEIPEAQIHEGFQEL | AAT |
| | LRTLNQPDSQLQLTTGNGLFLSEGLKLVDKFLEDVKKLYHSEAFTVNF | sequence 1 |
| | GDTEEAKKQINDYVEKGTQGKIVDLVKELDRDTVFALVNYIFFKGKWE | |
| | RPFEVKDATEEEDFHVDQVTTVKVPMMKRLGMFNIQHCKKLSSWVLL | |
| | MKYLGNATAIFFLPDEGKLQHLENELTHDIITKFLENEDRRSASLHLPKL | |
| | SITGTYDLKSVLGQLGITKVFSNGADLSGVTEEAPLKLSKAVHKAVLTID | |
| | EKGTEAAGAMFLEAIPMSIPPEVKFNKPFVFLMIEQNTKSPLFMGKVVN | |
| | PTQK | |
| 4 | GAGGACCCTCAAGGTGACGCCGCTCAAAAGACTGATACCTCGCAC | Codon |
| | CACGACCAAGACCACCCAACTTTTAACAAGATTACTCCTAATCTAGC | optimized |
| | TGAGTTCGCATTCTCTCTCTACAGACAACTCGCTCATCAGTCTAACT | AAT coding |
| | CTACGAACATTTTCTTCTCCCCAGTGTCCATTGCAACTGCTTTCGCC | sequence 2 |
| | ATGCTTTCTTTGGGTACTAAGGCTGACACACACGACGAGATTTTAG | |
| | AAGGATTGAACTTTAATCTTACCGAAATTCCTGAGGCCCAAATTCAC | |
| | GAAGGTTTTCAGGAGCTGCTTAGAACTCTTAACCAGCCAGATAGCC | |
| | AGCTACAACTTACTACTGGAAATGGACTATTCTTGAGTGAAGGTCTG | |
| | AAGCTAGTTGACAAATTCCTCGAAGATGTGAAAAAGTTGTACCATTC | |
| | TGAGGCTTTCACCGTCAACTTCGGTGACACAGAGGAGGCTAAAAAG | |
| | CAGATCAACGACTACGTTGAAAAGGGTACGCAAGGTAAAATCGTTG | |
| | ACCTCGTTAAAGAGCTTGATAGAGACACCGTATTTGCTCTTGTCAAT | |
| | TACATCTTCTTTAAAGGAAAGTGGGAGAGACCTTTCGAGGTCAAGG | |
| | ACACCGAGGAGGAAGATTTTCATGTAGATCAAGTCACCACTGTTAA | |
| | GGTTCCAATGATGAAGCGTCTCGGTATGTTTAACATCCAACACTGTA | |
| | AAAAGCTATCTTCTTGGGTCCTTCTGATGAAGTACCTGGGTAACGC | |
| | TACTGCAATCTTCTTCCTACCAGATGAAGGTAAGCTCCAGCACCTA | |
| | GAGAACGAACTTACCCACGACATCATTACTAAGTTCCTGGAAAACG | |
| | AAGACCGTAGATCCGCTTCCCTGCACCTCCCAAAACTGTCTATCAC | |
| | TGGTACCTATGACCTCAAGTCTGTTCTGGGTCAACTAGGTATAACC | |
| | AAAGTGTTTTCAAACGGCGCTGACCTCTCCGGTGTGACTGAAGAGG | |
| | CACCTCTCAAGCTGTCCAAGGCCGTTCACAAGGCAGTCCTTACCAT | |
| | TGATGAAAAGGGAACTGAAGCTGCCGGCGCTATGTTCCTAGAGGC | |
| | CATCCCAATGTCAATTCCTCCCGAGGTTAAGTTCAACAAGCCATTC | |
| | GTCTTCCTAATGATTGAACAAAACACAAAGTCTCCTCTGTTCATGGG | |
| | AAAGGTTGTTAACCCAACCCAAAAGTAATAG | |
| 5 | EDPQGDAAQKTDTSHHDQDHPTFNKITPNLAEFAFSLYRQLAHQSNST | Human |
| | NIFFSPVSIATAFAMLSLGTKADTHDEILEGLNFNLTEIPEAQIHEGFQEL | AAT |
| | LRTLNQPDSQLQLTTGNGLFLSEGLKLVDKFLEDVKKLYHSEAFTVNF | sequence 2 |
| | GDTEEAKKQINDYVEKGTQGKIVDLVKELDRDTVFALVNYIFFKGKWE | |
| | RPFEVKDTEEEDFHVDQVTTVKVPMMKRLGMFNIQHCKKLSSWVLLM | |
| | KYLGNATAIFFLPDEGKLQHLENELTHDIITKFLENEDRRSASLHLPKLSI | |
| | TGTYDLKSVLGQLGITKVFSNGADLSGVTEEAPLKLSKAVHKAVLTIDE | |
| | KGTEAAGAMFLEAIPMSIPPEVKFNKPFVFLMIEQNTKSPLFMGKVVNP | |
| | TQK | |

### DETAILED DESCRIPTION OF THE INVENTION

All cited documents of the patent and non-patent literature are hereby incorporated by reference in their entirety.

### Alpha-1 Antitrypsin (AAT):

Alpha-1 antitrypsin (also known as A1AT, AAT, PI, SERPINA1) is a ˜52 kDa glycoprotein that is one of the most abundant endogenous serine protease inhibitors (SERPIN superfamily). AAT is considered to be an acute phase protein, whereby AAT concentration can increase many fold upon acute inflammation.

The AAT encoding region is preferably any nucleic acid that encodes a naturally occurring or synthetic AAT protein sequence that exhibits AAT function, with reduced, the same, similar or increased activity compared to human AAT, or is functionally analogous to human AAT. The amino acid sequence of AAT is available under accession number 1313184B from the NCBI database. Corresponding nucleic acid sequences that encode AAT may be provided by one skilled in the art of molecular biology or genetics. The use of sequence variants of AAT that exhibit functional analogy or similarity to the unmodified human form of AAT is encompassed by the present invention.

One AAT coding sequence (CDS) is published at http://www.ncbi.nlm.nih.gov/nuccore/NM_000295.4 and is one preferred embodiment. This sequence comprises bases 262 to 1518 of the full sequence. SEQ ID NO 1 represent one exemplary AAT encoding sequence.

In some embodiments of the invention the CDS is codon optimized to increase protein production. The coding sequence after codon optimization preferably reads as in SEQ ID NO 2.

The nucleotide sequence according to SEQ ID NO 1 and/or 2 encodes a human AAT protein of the amino acid sequence according to SEQ ID NO 3.

In embodiments, a human AAT protein is employed according to SEQ ID NO 5, corresponding to a human AAT sequence as known in the art.

The invention therefore encompasses the use of a genetically modified yeast, preferably Pichia pastoris, expressing recombinant AAT as described herein, wherein the yeast comprises a nucleic acid molecule selected from the group consisting of:
a) a nucleic acid molecule comprising a nucleotide sequence that encodes AAT protein, such as according to SEQ ID NO 3 or 5, preferably by a nucleotide sequence according to SEQ ID NO 1 or 2,
b) a nucleic acid molecule which is complementary to a nucleotide sequence in accordance with a);
c) a nucleic acid molecule comprising a nucleotide sequence having sufficient sequence identity to be functionally analogous/equivalent to a nucleotide sequence according to a) or b), comprising preferably a sequence identity to a nucleotide sequence according to a) or b) of at least 70%, 75%, 80%, 85%, preferably 90%, more preferably 95%;
d) a nucleic acid molecule which, as a consequence of the genetic code, is degenerated into a nucleotide sequence according to a) through c); and/or
e) a nucleic acid molecule according to a nucleotide sequence of a) through d) which is modified by deletions, additions, substitutions, translocations, inversions and/or insertions and is functionally analogous/equivalent to a nucleotide sequence according to a) through d).

The invention therefore encompasses an AAT protein according to SEQ ID NO 3 or 5 or variants thereof, or the use of a genetically modified yeast, preferably Pichia pastoris as described herein, comprising a nucleotide sequence encoding an amino acid sequence, according to SEQ ID NO 3 or 5.

The invention encompasses further sequence variants of SEQ ID NO 3, in particular those of at least 70% sequence identity to SEQ ID NO 3 or 5, preferably at least 75%, 80%, 85%, 90%, or at least 95% sequence identity to SEQ ID NO 3 or 5. Such sequence variants are preferably functionally analogous or equivalent to the human AAT disclosed herein. Variations in the length of the protein are also encompassed by the invention, in cases where the functional equivalence of human AAT of SEQ ID NO 3 or 5 is maintained. Truncations or extensions in the length of the protein of, for example, up 50 amino acids, 40, 30, 20, or 10 amino acids may maintain AAT activity and are therefore encompassed in the present invention.

Functionally analogous sequences refer to the ability to encode a functional AAT gene product and to enable the same or similar functional effect as human AAT. AAT function may be determined by its ability to inhibit a variety of proteases in vitro, such as trypsin, or by its ability to inhibit neutrophil elastase (as described below). Appropriate assays for determining protease activity or for determining neutrophil elastase activity are known to a skilled person.

Protein modifications to the AAT protein, which may occur through substitutions in amino acid sequence, and nucleic acid sequences encoding such molecules, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function. In general, the non-polar amino acids Gly, Ala, Val, IIe and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

The terminology “conservative amino acid substitutions” is well known in the art, which relates to substitution of a particular amino acid by one having a similar characteristic (e.g., similar charge or hydrophobicity, similar bulkiness). Examples include aspartic acid for glutamic acid, or isoleucine for leucine. A conservative substitution variant will 1) have only conservative amino acid substitutions relative to the parent sequence, 2) will have at least 90% sequence identity with respect to the parent sequence, preferably at least 95% identity, 96% identity, 97% identity, 98% identity or 99% or greater identity; and 3) will retain neuroprotective or neurorestorative activity. In this regard, any conservative substitution variant of the above-described polypeptide sequences is contemplated in accordance with this invention. Such variants are considered to be “an AAT protein”.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs. Software such as BLAST or Clustal enable such sequence alignments and calculation of percent identity. As used herein, the percent homology between two sequences is equivalent to the percent identity between the sequences. Determination of percent identity or homology between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software (Altschul et al., 1990). In some embodiments, the percent homology oridentity can be determined along the full-length of the nucleic acid.

In embodiments, the AAT may comprise a 0 to 10 amino acid addition or deletion at the N and/or C terminus of a relevant sequence. This means that the polypeptide may have a) 0, 1, 2, 3, 4, 5, 6, 7, 8, 9 or 10 additional amino acids at its N terminus and 0, 1, 2, 3, 4, 5, 6, 7, 8, 9 or 10 amino acids deleted at its C terminus or b) 0, 1, 2, 3, 4, 5, 6, 7, 8, 9 or 10 additional amino acids at its C terminus and 0, 1, 2, 3, 4, 5, 6, 7, 8, 9 or 10 nucleotides deleted at its N terminus, c) 0, 1, 2, 3, 4, 5, 6, 7, 8, 9 or 10 additional amino acids at its N terminus and 0, 1, 2, 3, 4, 5, 6, 7, 8, 9 or 10 additional amino acids at its N terminus or d) 0, 1, 2, 3, 4, 5, 6, 7, 8, 9 or 10 amino acids deleted at its N terminus and 0, 1, 2, 3, 4, 5, 6, 7, 8, 9 or 10 amino acids deleted at its C terminus.

Furthermore, in addition to the polypeptides described herein, peptidomimetics are also contemplated. Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15:29; Veber and Freidinger (1985) TINS p. 392; and Evans et al. (1987) J. Med. Chem. 30:1229) and are usually developed with the aid of computerized molecular modelling. Peptide mimetics that are structurally similar to therapeutically useful peptides may be used to produce an equivalent therapeutic or prophylactic effect. It may be preferred in some embodiments to use peptide mimetics in order to prolong the stability of the polypeptides, when administered to a subject. To this end peptide mimetics for the polypeptides may be preferred that are not cleaved by human proteasomes.

A nucleic acid molecule encoding an AAT of the invention can be codon-optimized according to methods standard in the art for expression in the cell containing the target DNA of interest. For example, if the intended target nucleic acid is in a yeast cell, a yeast codon-optimized polynucleotide encoding AAT is contemplated for use in the constructs described herein.

In other embodiments, the Alpha-1 antitrypsin (AAT) protein may be replaced by other serine protease inhibitors. Any feature with reference to AAT as disclosed herein may therefore also relate to other serine protease inhibitors. In one embodiment, the serine protease inhibitor is a trypsin-like serine protease inhibitor.

A trypsin-like serine protease inhibitor inhibits a serine protease with trypsin-like activity. Serine serves as the nucleophilic amino acid at the enzyme's active site of the serine protease enzyme. Numerous trypsin-like serine proteases have been under active pursuit as therapeutic targets for inhibition. Viral entry via endocytosis is dependent on target cell proteases, such as serine proteases.

Viral infections are target cell serine protease dependent if the virus requires a target cell serine protease for viral entry and growth.

The target cell serine protease activates the virus Spike protein required for fusion of the viral and target cell membranes and the release of the viral genome into the cytosol of the target cell.

Targeting the host cell serine protease can prevent viral growth of an endocytosis dependent virus, whereby inhibiting the target cell serine protease is an effective method for treating a subject infected with the endocytosis dependent virus. The serine protease inhibitor, preferably a trypsin-like serine protease inhibitor, inhibits viral infection and virus growth. As non-limiting examples, trypsin-like serine protease inhibitors include camostat, aprotinin, benzamidine, gabexate, leupeptin, nafamostat, pepstatin A, ribavirin, sepimostat, ulinastatin, and patamostat.

### Molecules:

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, an AAT gene or portion thereof, or regulatory elements. A nucleic acid molecule can be single-stranded or double-stranded.

An “AAT nucleic acid” or refers to a nucleic acid that comprises the AAT gene or a portion thereof, or a functional variant of the AAT gene or a portion thereof. A functional variant of a gene includes a variant of the gene with minor variations such as, for example, silent mutations, single nucleotide polymorphisms, missense mutations, and other mutations or deletions that do not significantly alter gene function.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular AAT protein (including fragments and portions thereof) is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

As used herein, “protein” or “polypeptide” shall mean both peptides and proteins. In this invention, the polypeptides may be naturally occurring or recombinant (i.e., produced via recombinant DNA technology), and may contain mutations (e.g., point, insertion and deletion mutations) as well as other covalent modifications (e.g., glycosylation and labelling (via biotin, streptavidin, fluorescein, and radioisotopes)) or other molecular bonds to additional components. For example, PEGylated proteins are encompassed by the scope of the present invention. PEGylation has been widely used as a post-production modification methodology for improving biomedical efficacy and physicochemical properties of therapeutic proteins. Applicability and safety of this technology have been proven by use of various PEGylated pharmaceuticals for many years (refer Jevsevar et al, Biotechnol J. 2010 January; 5 (1): 1 13-28). In some embodiments the polypeptides described herein are modified to exhibit longer in vivo half-lives and resist degradation when compared to unmodified polypeptides. Such modifications are known to a skilled person, such as cyclized polypeptides, polypeptides fused to Vitamin B12, stapled peptides, protein lipidization and the substitution of natural L-amino acids with D-amino acids (refer Bruno et al, Ther Deliv. 2013 November; 4 (11): 1443-1467).

### Producing Recombinant AAT in Yeast

The invention relates to a method of producing recombinant AAT or fragment(s) thereof from genetically modified yeast, comprising the steps of culturing a genetically modified yeast comprising an exogenous nucleic acid molecule with an AAT-encoding region operably linked to a promoter or promoter/enhancer combination, expressing recombinant AAT in the cultured yeast, and isolating recombinant AAT from the culture.

Yeast expression platform that can be used in the context of the invention comprise a strain of yeast used to produce large amounts of proteins, here AAT, for research or industrial uses. While yeast are often more resource-intensive to maintain than for example bacteria, certain products can only be produced by eukaryotic cells like yeast, necessitating use of a yeast expression platform. Yeasts differ in productivity and with respect to their capabilities to secrete, process and modify proteins. As such, different types of yeast (i.e. different expression platforms) are better suited for different research and industrial applications. Importantly, protein expression and production in yeast is advantageous since yeast is able to grow rapidly in large containers, produce proteins in an efficient way, is safe and can produce and modify the protein products to be as ready for human consumption in the context of cosmetic compositions.

The manufacture of recombinant therapeutics is a fast-developing section of therapeutic pharmaceuticals. Yeasts are established eukaryotic host for heterologous protein production and offer distinctive benefits in synthesising pharmaceutical recombinants. Yeasts are proficient of vigorous growth on inexpensive media, easy for gene manipulations, and are capable of adding post translational changes of eukaryotes. Numerous yeasts comprising Saccharomyces cerevisiae, Pichia pastoris, Hansenula polymorpha, Yarrowia lipolytica, Arxula adeninivorans, Kluyveromyces lactis, and Schizosaccharomyces pombe are exemplary yeast hosts for recombinant protein production, and may be employed accordingly.

Yeasts are common hosts for the production of proteins from recombinant DNA. They offer relatively easy genetic manipulation and rapid growth to high cell densities on inexpensive media. As eukaryotes, they are able to perform protein modifications like glycosylation which are common in eukaryotic cells, but relatively rare in bacteria. Due to this, yeast can produce complex proteins that are identical or very similar to native products from mammals and in particular humans. The most commonly used yeast expression platform is based on the baker's yeast Saccharomyces cerevisiae. However, further yeast expression platforms such as Saccharomycesaceae Pichia, in particular, Pichia pastoris have been studied and are widely used for various applications based on their different characteristics and capabilities. For instance, some of them grow on a wide range of carbon sources and are not restricted to glucose, as it is the case with baker's yeast. Several of them are also applied to genetic engineering and to the production of foreign proteins and can be used in the context of the present invention.

In embodiments, the yeast related to the present invention is of the family Saccharomycesaceae, preferably Saccharomycesceae Pichia, more preferably Pichia pastoris.

Pichia pastoris is an excellent expression host for the production of heterologous proteins including industrial enzymes and biopharmaceuticals. So far, this methylotrophic expression system has been successfully utilized for the generation of numerous recombinant proteins including human erythropoietin, phospholipase C, phytase, human superoxide dismutase, trypsin, human serum albumin, collagen and human monoclonal antibody 3H6 Fab fragment. Compared to any other yeast species, P. pastoris is more efficient in the secretory production of recombinant proteins. The industrial interest to this host is also attributed to powerful methanol-regulated alcohol oxidase promoter (AOX1), highly efficient secretion mechanism, posttranslational modification capabilities, and high cell density growing on defined medium. Various recombinant proteins have been expressed using P. pastoris. There are several successful cases for production of therapeutic protein in P. pastoris. In a previous study (2016), it has been demonstrated the production of a nanobody (VHH) against Clostridium botulinum neurotoxin type E (BoNT/E) in P. pastoris. A product yield of 16 mg/l was achieved that was higher than the levels produced by E. coli. In addition, Xia et al. showed production of 30 mg/l of recombinant angiogenin in this yeast. Furthermore, the productivity of 111 mg/l for human adiponectin, 8.1 g/l for recombinant xylanase and 260 mg/l of anti-HIV antibody have been reported in P. pastoris.

The average concentration of AAT in plasma is 1.3 mg/ml, with a half-life of 3 to 5 days. Protein size, glycosylation pattern, metastable inhibitory nature of AAT and production cost represent the challenges in the production of recombinant AAT. The methylotrophic yeast P. pastoris as an attractive host for production of human AAT. It owns the ability for introducing many of post-translational modifications such as glycosylation and proteolytic processing which is obtained through moving in the secretory pathway. For glycoproteins such as AAT, these modifications are very important for appropriate function and/or structure.

In embodiments, the AAT is expressed in P. pastoris as a fusion protein to a histidine tag (His-tag) which facilitates purification step. Signal sequence from Saccharomyces cerevisiae α-factor secretion signal was included to direct secretion of the protein to extracellular medium. This powerful expression system made use of the highly inducible alcohol oxidase 1 (AOX1) promoter to express large quantities of glycosylated protein. The P. pastoris produced AAT activity and its features were evaluated using elastase inhibitory assay, respectively.

In embodiments, the genetically modified yeast is of the family Saccharomycesceae, preferably Saccharomycesaceae Saccharomyces, more preferably Saccharomyces cerevisiae. In embodiments, S. cerevisiae may be a preferred host over bacteria. The Saccharomyces cerevisiae expression system is one of the most commonly used eukaryotic organisms that has been utilized as a model to study various biological phenomena and for the recombinant production of therapeutic proteins. Potential issues in protein glycosylation using Saccharomyces cerevisiae have been addressed, making S. cerevisiae a viable yeast for therapeutic protein production. For example, it has been shown that disruption of Mnn2p and Mnn11p genes that are related to glycosylation modification pathways, improved the production of recombinant cellulases. Furthermore, removal of the α-1,6-mannosyltransferase Och1p enhanced the production of active form of human tissue-type plasminogen activator. These studies indicated that N-glycosylation modification also leads to increased protein secretion. Recombinant proteins can be expressed intracellularly or directed to the secretory apparatus using a secretory signal peptide. The frequently used signal sequence that is functional in all yeast expression systems, is prepro-sequence of mating factor α1 (MFα1).

Saccharomyces cerevisiae has also some important advantages from the safety point of view and this property encourages the use of this system in various industrial processes. It is generally regarded as safe (GRAS) because S. cerevisiae is nonpathogenic and historically have used in various nutritional industries and production of biopharmaceuticals. On the other hand, current knowledge on genetics, physiology, and fermentation of yeasts facilitate the use of this organism in the production of useful products. Hepatitis B surface antigen, hirudin, insulin, glucagon, urate oxidase, macrophage colony-stimulating factor and platelet-derived growth factor are examples of products on the market from S. cerevisiae. Alternative expression systems including methylotrophic yeasts Pichia pastoris and Hansenula polymorpha and non-methylotrophic yeast Yarrowia lipolytica, Kluyveromyces lactis and Arxula adeninivorans are also viable alternatives.

### Post-Translational Modification

In one embodiment, the method as used herein comprises post-translational modification of the recombinant AAT protein.

In embodiments, the post-translational modification is O-glycosylation, N-glycosylation, N-terminal methionine removal, N-acetylation and/or phosphorylation or any combination thereof.

In embodiments, the recombinant AAT protein has one or more human-like glycoform patterns.

In embodiments, the method as used herein comprises modifying the recombinant AAT protein in vitro after isolation from the yeast.

In some embodiments, the modification is covalent attachment of recombinant AAT protein to a biocompatible polymer.

The term “Posttranslational modification” refers to any alteration of the translated polypeptide chain of AAT that occurs after or during translation. This includes modifications of amino acid side chains of the polypeptide or modifications of the terminal amino or carboxyl group. Posttranslational modification refers to attaching a biochemical group such as acetate, phosphate, carbohydrate moieties and lipids to amino acid side chains that alter the biochemical and physical properties of a protein after translation. Many proteins undergo post translational modifications shortly after their translation and some after protein folding and some after localization. Most common protein post translational modifications are Phosphorylation, Glycosylation, Methylation, Ubiquitination, S-Nitrosylation, N-Acetylation.

Such modifications can be covalent modifications carried out enzymatically by the expression system following protein biosynthesis. Posttranslational modifications further comprise enzymatic cleavage of peptide bonds and processing of the translated protein, covalent additions of particular chemical groups, polymer, lipids, carbohydrates, or even entire proteins to amino acid side chains. These chemical modifications of a polypeptide chain after its biosynthesis extends the range of amino acid structures and properties, and consequently, diversifies structures and functions of proteins. In a preferred embodiment, the recombinant AAT protein undergoes posttranslational modification, such as phosphorylation, glycosylation, ubiquitination, nitrosylation, methylation, acetylation, lipidation and proteolysis.

In one aspect of the invention, secreted or released AAT protein undergoes glycosylation encompassing a diverse selection of sugar-moiety additions to the proteins that ranges from simple monosaccharide modification of nuclear transcription factors to highly complex branched polysaccharide changes of the cell surface receptors in the yeast expression system, preferably O-glycosylation and/or N-glycosylation. N-glycosylation is binding of carbohydrates to asparagine of the polypeptide. O-glycosylation is binding of carbohydrates to serine/threonine.

In embodiments, a modification of recombinant AAT protein is the covalent attachment of recombinant AAT to a biocompatible polymer such as polyethylene glycol (PEG). The term “PEGylation” as used herein refers to a process of both covalent and non-covalent attachment of polyethylene glycol polymer chains to molecules and macrostructures, such as a drug, or a bioactive protein or vesicle. PEGylation is routinely performed through incubation of a reactive derivative of PEG with the target molecule. The covalent attachment of PEG to a protein can “mask” the agent from the host's immune system (reducing immunogenicity and antigenicity), and increase its hydrodynamic size (size in solution), which prolongs its circulatory time by reducing renal clearance. In one aspect, PEGylation can occur at N-terminus of the recombinant AAT protein.

As used herein, the term “biocompatible polymer” refers to polymer with the suitability to body and body fluids exposure. Biocompatible polymers are both synthetic and natural and aid in the close vicinity of a living system or work in intimacy with living cells. These are used to gauge, treat, boost, or substitute any tissue, organ or function of the body. A biocompatible polymer improves body functions without altering its normal functioning and triggering allergies or other side effects. It encompasses advances in tissue culture, tissue scaffolds, implantation, artificial grafts, wound fabrication, controlled drug delivery, bone filler material, etc. Biocompatible polymer are such as, Polystyrene (PS), Polypropylene (PP), Polyvinyl chloride (PVC), Polyethylene (PE), Polyurethane (PU), Polycarbonate (PC), Polyethylene terephthalate (PET), Polyetheretherketone (PEEK).

As used herein, “polyethylene glycol” is a polyether compound with many applications, from industrial manufacturing to medicine. PEG is also known as polyethylene oxide or polyoxyethylene, depending on its molecular weight.

In certain aspects, a recombinant AAT protein is conjugated to a water-soluble polymer. This can occur by any of a variety of chemical methods known in the art. For example, in one embodiment the recombinant AAT protein is modified by the conjugation of PEG to free amino groups of the protein using N-hydroxysuccinimide (NHS) esters. In another embodiment the water-soluble polymer, for example PEG, is coupled to free SH groups using maleimide chemistry or the coupling of PEG hydrazides or PEG amines to carbohydrate moieties of the recombinant AAT protein after prior oxidation.

According to one embodiment of the present invention, PEGylation is performed using PEG of 20 kDa or more. PEG is conjugated to AAT and provides an effect of wrapping around the protein, which leads to the defense against the loss after binding to a scavenger receptor or degradation by an inactivating protease.

PEG is a polymer in a chain form that is linear or branched, and PEG having small molecular weight cannot fully wrap the protein and thus cannot fully protect the protein. Thus, the molecular weight should be the same or above the critical level to sufficiently wrap the protein to be protected. PEGylation of the recombinant AAT protein of the present invention can be carried out by substituting the amino acid at the PEGylation position with cysteine and then conjugating to the PEG containing an acryloyl, sulfone or maleimide group specific to cysteine at one end.

Glycosylated AAT Produced from Yeast

In embodiments, the post-translational modification is O-glycosylation, N-glycosylation, N-terminal methionine removal, N-acetylation and/or phosphorylation or any combination thereof. In embodiments, the recombinant AAT protein has one or more human-like glycoform patterns.

Yeast for producing AAT as used herein allows the AAT to be modified into human-like structures. The term “human-like” structure or “human-like” glycoform patterns are related to the glycosylation pattern, which e.g. extends the serum half-life of the recombinant AAT and/or stability comparable to human plasma derived AAT. “Human-like” glycoform patterns are preferably different from but similar to human glycosylation patterns, to an extent of extending the serum half-life of the recombinant AAT compared to non-modified AAT protein. “Human-like” glycoform patterns may, by way of example, comprise one or more glycans selected from the group consisting of mannose, N-acetylglucosamine (GlcNAc), galactose, N-Acetylneuraminic acid (Neu5Ac), N-Glycolylneuraminic acid and Xylose.

The glycosylation pattern of recombinant AAT by yeast and other post-translational modification by yeast renders a new type of recombinant AAT. This can be verified by an SDS-page analysis by skilled person. A shift up or down of the band from recombinant AAT produced from yeast, such as Pichia pastoris compared to the band of plasma derived AAT can be an evident of new type of recombinant AAT.

### Recombinant AAT Isolation/Purification:

According to the present invention, AAT is preferably purified or isolated, for example from a culture supernatant or other preparation obtained from yeast culture, using means known to a skilled person. The harvest of material obtained by fermentation of Pichia pastoris is possible using standard laboratory equipment, in order to process products from a supernatant. For example, a suitable process comprises of a centrifugation step and/or depth filtration, followed optionally by a (sterile) membrane filtration. Also available is a low-speed centrifugation, sufficient to pellet down the yeast cells, leaving a clear and transparent supernatant, which also may serve as evidence for the absence of bacterial contamination. Such procedures yield supernatant ready for follow-on purification steps, such as chromatography.

For example, various methods involving liquid chromatography, suitable to separate a protein of interest, may be elected by a skilled person without undue effort. For example, solid phases used for chromatography, known as chromatography media or resins, are typically engineered porous inert supports functionalized with various chemical groups that determine the interactions with the molecules to be separated. Commonly used modes of separation, applicable for the rAAT described herein, are, without limitation, based on specific binding interactions (affinity chromatography), charge (ion exchange chromatography), size (size exclusion chromatography/gel filtration chromatography), hydrophobic surface area (hydrophobic interaction chromatography and reverse phase chromatography) and/or multiple properties (multimodal or mixed-mode chromatography).

### Medical Indications:

In one aspect of the present invention there is provided a pharmaceutical composition or combination as herein described for use in the treatment and/or prevention of a viral infection in a subject and/or a medical condition associated with a viral infection. Preferred viral infections to be treated or associated diseases are those described herein.

As used herein, “viral infection” occurs when an organism's body is invaded by pathogenic viruses. The virus type is preferably selected from the group consisting of adenovirus, coxsackievirus, Epstein-Barr virus, hepatitis A virus, hepatitis B virus, hepatitis C virus, Herpes simplex virus type 1, Herpes simplex virus type 2, cytomegalovirus, human Herpes virus type 8, HIV, RSV, influenza virus, measles virus, mumps virus, human papillomavirus, poliovirus, rabies virus, respiratory syncytial virus, rubella virus, varicella-zoster virus, SARS-CoV-2 virus.

In one embodiment, the family of the virus is preferably selected from the group of adenoviridae, picornaviridae, Herpesviridae, picornaviridae, hepadnaviridae, Flaviviridae, Herpesviridae, retroviridae, Orthomyxoviridae, paramyxoviridae, papillomavirus, picornaviridae, Rhaboviridae, Togaviridae, Herpesviridae and coronaviridae.

By way of examples, respiratory virus disease may occur from any infection by an adenovirus, coronaviruses (common cold viruses), influenza (flu), parainfluenza, parvovirus b19 (fifth disease), respiratory syncytial virus (RSV) or rhinovirus (common cold). Further viral respiratory disease or virus may relate to HBoV, human bocavirus; HCoV, human coronavirus; HMPV, human metapneumovirus; HPIV, human parainfluenza virus; HRSV, human respiratory syncytial virus; HRV, human rhinovirus; PCF, pharyngoconjunctival fever; SARS, severe acute respiratory syndrome; SARS-CoV, Coronavirus associated with SARS; URI, upper respiratory infection.

Any one or more of the above-mentioned viruses may be susceptible to the inventive treatment described herein and the treatment of each virus as such represents an embodiment of the present invention.

In embodiments, the invention also relates to a recombinant alpha1-antitrysin (AAT) protein or a fragment thereof expressed in a genetically modified yeast for use in the treatment or prevention of one or more of the above viral infections.

In embodiments, the invention also relates to a recombinant alpha1-antitrysin (AAT) protein or a fragment thereof expressed in a genetically modified yeast for use in the treatment or prevention of an influenza infection.

In one aspect of the present invention there is provided a pharmaceutical composition or combination according to the invention as described herein for use in the treatment of a medical condition associated with a coronavirus, such as a SARS Coronavirus, wherein the medical condition associated with a SARS Coronavirus is preferably COVID-19 or a SARS Coronavirus-associated respiratory disease.

As used herein, the “patient” or “subject” may be a vertebrate. In the context of the present invention, the term “subject” includes both humans and animals, particularly mammals, and other organisms.

As used herein, “a subject in need thereof,” refers to a subject afflicted with, or at risk of developing, a disease, specifically, coronavirus disease. The subject in need thereof may be a subject being hospitalized with symptoms of coronavirus disease, a subject having symptoms of coronavirus diseases and is under ambulatory setting or at home, or a subject having asymptomatic coronavirus disease and is under ambulatory setting or at home.

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

In one embodiment, the treatment described herein relates to either reducing or inhibiting Coronavirus infection or symptoms thereof via preventing viral entry into target cells. The prophylactic therapy as described herein is intended to encompass prevention or reduction of risk of Coronavirus infection, due to a reduced likelihood of Coronavirus infection of cells via interaction with the ACE2 protein after treatment with the agents described herein.

As used herein, a “patient with symptoms of an infectious disease” is a subject who presents with one or more of, without limitation, fever, diarrhea, fatigue, muscle aches, coughing, if have been bitten by an animal, having trouble breathing, severe headache with fever, rash or swelling, unexplained or prolonged fever or vision problems. Other symptoms may be fever and chills, very low body temperature, decreased output of urine (oliguria), rapid pulse, rapid breathing, nausea and vomiting. In preferred embodiments the symptoms of an infectious disease are fever, diarrhea, fatigue, muscle aches, rapid pulse, rapid breathing, nausea and vomiting and/or coughing.

As used herein, a patient with “symptoms of a viral infection of the respiratory tract” is a subject who presents with one or more of, without limitation, cold-like symptoms or flu-like illnesses, such as fever, cough, runny nose, sneezing, sore throat, having trouble breathing, headache, muscle aches, fatigue, rapid pulse, rapid breathing, nausea and vomiting, lack of taste and/or smell and/or malaise (feeling unwell).

In some embodiments, symptoms of infection with a SARS-virus are fever, sore throat, cough, myalgia or fatigue, and in some embodiments, additionally, sputum production, headache, hemoptysis and/or diarrhea. In some embodiments, symptoms of an infection with a SARS-coronavirus, for example SARS-CoV-2, are fever, sore throat, cough, lack of taste and/or smell, shortness of breath and/or fatigue.

As used herein, the term “a patient that is at risk of developing a severe acute respiratory syndrome (SARS)” relates to a subject, preferably distinct from any given person in the general population, who has an increased (e.g. above-average) risk of developing SARS. In some embodiments, the patient has symptoms of SARS or symptoms of a SARS Coronavirus infection. In some embodiments, the patient has no symptoms of SARS or symptoms of a SARS Coronavirus infection. In some embodiments, the subject has been in contact with people with SARS Coronavirus infections or symptoms. In some embodiments, the person at risk of developing SARS has been tested for the presence of a SARS Coronavirus infection. In some embodiments, the person at risk of developing SARS has tested positive for the presence of a SARS Coronavirus infection, preferably a coronavirus infection.

In embodiments, the patient at risk of developing SARS is an asymptomatic patient that shows no specific symptoms of SARS (yet). An asymptomatic patient may be at risk of developing SARS because the patient has been in contact with a person infected with a SARS Coronavirus. For example, the asymptomatic patient may have been identified as being at risk of developing SARS by a software application (app) that is installed on his smart phone or corresponding (portable) device and that indicates physical proximity or short physical distance to an infected patient that uses a corresponding app on its respective mobile device/smart phone. Other methods of determining contact/physical proximity to an infected person are known to the skilled person and equally apply to the method of the invention.

In some embodiments, the patient that has or is at risk of developing a severe acute respiratory syndrome (SARS) has a coronavirus infection.

Coronaviruses are a group of related viruses that cause diseases in mammals and birds. The scientific name for coronavirus is Orthocoronavirinae or Coronavirinae. Coronavirus belongs to the family of Coronaviridae. The family is divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts.

In humans, coronaviruses cause respiratory tract infections that can be mild, such as some cases of the common cold, and others that can be lethal, such as SARS, MERS, and COVID-19. Coronaviruses are enveloped viruses with a positive-sense single-stranded RNA genome and a nucleocapsid of helical symmetry. The genome size of coronaviruses ranges from approximately 27 to 34 kilobases, the largest among known RNA viruses.

Various species of human coronaviruses are known, such as, without limitation, Human coronavirus OC43 (HCoV-OC43), of the genus β-CoV, Human coronavirus HKU1 (HCoV-HKU1), of the genus β-CoV, Human coronavirus 229E (HCoV-229E), α-CoV, Human coronavirus NL63 (HCoV-NL63), α-CoV, Middle East respiratory syndrome-related coronavirus (MERS-CoV), Severe acute respiratory syndrome coronavirus (SARS-CoV), Severe acute respiratory syndrome coronavirus 2 (SARS-CoV-2).

Coronaviruses vary significantly in risk factor. Some can kill more than 30% of those infected (such as MERS-CoV), and some are relatively harmless, such as the common cold. Coronaviruses cause colds with major symptoms, such as fever, and a sore throat, e.g. from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). Coronaviruses can also cause SARS.

Advances in nucleic acid sequencing technology (commonly termed Next-Generation Sequencing, NGS) are providing large sets of sequence data obtained from a variety of biological samples and allowing the characterization of both known and novel virus strains. Established methods are therefore available for determining a Coronavirus infection.

As used herein the “Long COVID” also known as post-COVID-19 syndrome, post-acute sequelae of COVID-19 (PASC), or chronic COVID syndrome (CCS), shall mean a condition characterized by long-term sequelae appearing or persisting after the typical convalescence period of COVID-19. Long COVID can affect nearly every organ system, with sequelae including respiratory system disorders, nervous system and neurocognitive disorders, mental health disorders, metabolic disorders, cardiovascular disorders, gastrointestinal disorders, malaise, fatigue, musculoskeletal pain, and anemia. A wide range of symptoms are commonly reported, including fatigue, headaches, shortness of breath, anosmia (loss of smell), parosmia (distorted smell), muscle weakness, low fever and cognitive dysfunction.

Further symptoms or medical condition associated with Long COVID include but not limited to extreme fatigue, long lasting cough, muscle weakness, low grade fever, inability to concentrate (brain fog), memory lapses, changes in mood, sometimes accompanied by depression and other mental health problems sleep difficulties, headaches, joint pain, needle pains in arms and legs, diarrhoea and bouts of vomiting, loss of taste and smell, sore throat and difficulties swallowing, new onset of diabetes and hypertension, heartburn (gastroesophageal reflux disease), skin rash, shortness of breath, chest pains, palpitations, kidney problems (acute kidney injury, and chronic kidney disease), changes in oral health (teeth, saliva, gums), anosmia (lack of sense of smell), parosmia (change in sense of smell), tinnitus, blood clotting (deep vein thrombosis and pulmonary embolism)

In one embodiment, long COVID for new or ongoing symptoms 4 weeks or more after the start of acute COVID-19, which is divided into the other two: ongoing symptomatic COVID-19 for effects from 4 to 12 weeks after onset, and post-COVID-19 syndrome for effects that persist 12 or more weeks after onset.

In other embodiment, Long COVID symptoms for individuals who “don't recover fully over a period of a few weeks”, be collectively referred to as Post-Acute Sequelae of SARS-CoV-2 infection (PASC). The symptoms of Long COVID includes fatigue, shortness of breath, “brain fog”, sleep disorders, intermittent fevers, gastrointestinal symptoms, anxiety, and depression. Symptoms can persist for months and can range from mild to incapacitating, with new symptoms arising well after the time of infection. The CDC term Post-Covid Conditions qualifies Long Covid as symptoms 4 or more weeks after first infection.

### Compositions and Administration:

The AAT protein or composition comprising said protein as described herein may comprise different types of carriers depending on whether they are to be administered in solid, liquid or aerosol form, and whether they need to be sterile for such routes of administration as injection.

The active agent AAT can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, directly, via a catheter, via a lavage, in cremes, in lipid compositions (e.g., liposomes), locally applied by sponges or by other method or any combination of the forgoing as would be known to one of ordinary skill in the art (see, for example, Remington's Pharmaceutical Sciences, 18th Ed. Mack Printing Company, 1990, incorporated herein by reference).

The present invention encompasses treatment of a patient by introducing a therapeutically effective number polypeptides into a subject, or a subject's bloodstream. As used herein, “introducing” polypeptides into the subject's bloodstream shall include, without limitation, introducing such polypeptides into one of the subject's veins or arteries via injection. Such administering can also be performed, for example, once, a plurality of times, and/or over one or more extended periods. A single injection is preferred, but repeated injections over time (e.g., quarterly, half-yearly or yearly) may be necessary in some instances. Such administering is also preferably performed using an admixture of polypeptides and a pharmaceutically acceptable carrier. Pharmaceutically acceptable carriers are well known to those skilled in the art and include, but are not limited to, 0.01-0.1 M and preferably 0.05 M phosphate buffer or 0.8% saline.

As used herein, the term “therapeutically effective amount” is exchangeable with any one of “therapeutically effective dose” or “sufficient/effective amount or dose,” and refers to a dose that produces the required therapeutic effects. Specifically, an effective dose generally refers to the amount of the composition disclosed herein sufficient to induce immunity, to prevent and/or ameliorate coronavirus infection, or to reduce at least one symptom associated with the coronavirus infection and/or to enhance the efficacy of another therapeutic composition. An effective dose may refer to the amount of the composition sufficient to delay or minimize the onset of an infection. An effective dose may refer to the amount of the composition sufficient to prevent infection by a virus or reduce risk of infection by a virus. An effective dose may also refer to the amount of the composition that provides a therapeutic benefit in the treatment or management of infection. In addition, an effective dose may be the amount with respect to the composition alone, or in combination with other therapies, that provides a therapeutic benefit in the treatment or management of a viral infection. An effective dose may also be the amount sufficient to enhance a subject's (in particular, human's) own immune response against a subsequent exposure to coronavirus. The exact effective dose depends on the purpose of the treatment, and is ascertainable by one skilled in the art using known techniques.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Additionally, such pharmaceutically acceptable carriers can be aqueous or non-aqueous solutions, suspensions, and emulsions, most preferably aqueous solutions. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as Ringer's dextrose, those based on Ringer's dextrose, and the like. Fluids used commonly for i.v. administration are found, for example, in Remington: The Science and Practice of Pharmacy, 20th Ed., p. 808, Lippincott Williams S-Wilkins (2000). Preservatives and other additives may also be present, such as, for example, antimicrobials, antioxidants, chelating agents, inert gases, and the like.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form in accordance with this invention can be in the form of a reconstitutable lyophilizate comprising a dose of a composition comprising granulin. In one aspect of the present embodiment, any of a number of prolonged or sustained release dosage forms known in the art can be administered such as, for example, the biodegradable carbohydrate matrices described in U.S. Pat. Nos. 4,713,249; 5,266,333; and 5,417,982, the disclosures of which are incorporated herein by reference.

In an illustrative embodiment pharmaceutical formulations for general use with AAT for parenteral administration comprising: a) a pharmaceutically active amount of the granulin; b) a pharmaceutically acceptable pH buffering agent to provide a pH in the range of about pH 4.5 to about pH 9; c) an ionic strength modifying agent in the concentration range of about 0 to about 250 millimolar; and d) water soluble viscosity modifying agent in the concentration range of about 0.5% to about 7% total formula weight are described or any combinations of a), b), c) and d).

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, Cacodylate, MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Compositions of the present invention are preferably formulated for parenteral administration, e.g., intravascular (intravenous or intraarterial), intraperitoneal or intramuscular administration. The term “parenteral administration” refers to a mode of administration, such as by injection or infusion, other than through the alimentary canal and further refers to subcutaneous, intramuscular, or intravenous injection, intraperitoneal injection, which is also contemplated herein. Intramuscular administration includes intravenous or intraarterial administration. The liquid pharmaceutical compositions, whether they be solutions, suspensions or other like form, may include one or more of the following suitable excipients: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile. In addition, the pharmaceutical compositions to be administered may also contain, if desired, small amounts of nontoxic auxiliary substances such as wetting or emulsifying agents, pH buffering agents, stabilizers, dissolution enhancers and other such agents, for example, sodium acetate, sorbitan monolaurate, triethanolamine oleate and cyclodextrins.

The unitary daily dosage of the composition comprising the AAT protein can vary significantly depending on the patient condition, the disease state being treated, the route of administration of AAT and tissue distribution, and the possibility of co-usage of other therapeutic treatments. The effective amount of AAT to be administered to the patient is based on body surface area, patient weight, physician assessment of patient condition, and the like.

In one illustrative embodiment, an effective dose of AAT can range from about 1 mg/kg of patient body weight to about 500 mg/kg of patient body weight, more preferably from about 10 mg/kg of patient body weight to about 300 mg/kg of patient body weight, and more preferably from about 50 mg/kg of patient body weight to about 200 mg/kg of patient body weight, for example about 100 mg/kg or about 180 mg/kg body weight.

According to the invention, administration of the composition may preferably occur through inhalation to a mucosal surface of the respiratory tract a subject. “Inhalation administration” refers to both mouth inhalation and nasal inhalation. “Mouth inhalation” relates to drugs administered by inhalation through the mouth and must be atomized into smaller droplets than those administered by the nasal route, so that the drugs can pass through the windpipe (trachea) and into the lungs.

How deeply into the lungs they go depends on the size of the droplets. Smaller droplets go deeper, which increases the amount of drug absorbed. Inside the lungs, they are absorbed into the bloodstream.

The drug administered by this route can be delivered by a metered-dosed container (inhaler) in form of dry powder.

The drug can also be nebulized from a solution comprising said drug into mist or into suitable-sized droplets or aerosol by a nebulizer, such as Pari Boy Pro for inhalation.

The inhaled medications can be absorbed quickly and act both locally and systemically. An inhaler device can achieve the correct dose. In general, only 20-50% of the pulmonary-delivered dose rendered in powdery particles will be deposited in the lung upon mouth inhalation. The remainder of 50-70% undeposited aerosolized particles are cleared out of lung as soon as exhalation. In embodiments an inhaled powdery particle that is >8 μm is structurally predisposed to depositing in the central and conducting airway by inertial impaction. In embodiments, an inhaled powdery particle that is between 3 and 8 μm in diameter tend to largely deposit in the transitional zones of the lung by sedimentation. In embodiments, an inhaled powdery particle that is <3 μm in diameter is structurally predisposed to depositing primarily in the respiratory regions of the peripheral lung via diffusion.

The term “nasal inhalation” refers to a drug product or preparation, including the delivery device if applicable, whose intended site of deposition is the respiratory tract or the nasal or pharyngeal region. Nasal inhalation does not include a topical nasal spray or irrigation that is deposited primarily in the nasal passages. The drug can also be nebulized from a solution comprising said drug into mist or into suitable-sized droplets or aerosol by a nebulizer such as Pari Boy Pro for inhalation.

The simultaneous administration and/or subsequential administration of the pharmaceutical composition by different administration routes are also contemplated in the context of the invention. In embodiments, the administration route of the composition comprises inhalation administration. In a preferred embodiment, parenteral administration of the pharmaceutical composition according to the present invention or a pharmaceutical composition comprising a human plasma derived AAT protein is carried out simultaneously or subsequentially. In another preferred embodiment, nasal and/or oral administration of pharmaceutical composition according to the present invention or a pharmaceutical composition comprising a human plasma derived AAT protein is carried out simultaneously or subsequentially.

As used herein, the “respiratory tract” is commonly considered to comprise the nose, pharynx, larynx, trachea, and the lung with its different compartments. The respiratory tract is involved with the process of respiration in mammals, and is a part of the respiratory system, and is lined with respiratory mucosa or respiratory epithelium.

As used herein, a “mucosal surface” is characterised by the presence of an overlying mucosal fluid, e.g. saliva, tears, nasal, gastric, cervical and bronchial mucus, the functions of which include to supply and deliver an array of immunoregulatory and pro-healing species including growth factors, antimicrobial proteins and immunoglobulins.

As used herein, a “pharmaceutical excipient” is essentially anything other than the active pharmaceutical ingredient in a composition.

In the formulation of pharmaceutical compositions, excipients are generally added along with the active pharmaceutical ingredients in order to 1) protect, support or enhance the stability of the formulation; 2) bulk up the formulation in case of potent drug for assisting in the formulation of an accurate dosage form; 3) improve patient acceptance; 4) help improve bioavailability of active drugs; 5) enhance overall safety and effectiveness of the formulation during its storage and use.

Excipients used in the formulation of pharmaceutical compositions are sub-divided into various functional classifications, depending on the role they intend to play in the resultant formulation. Some excipients can have different functional roles in different formulation types and in addition, individual excipients can have different grades, types, and sources depending on those different functional roles. The possible types of excipients commonly used in the formulation of pharmaceutical suspensions include solvents/vehicle, co-solvent, buffering agents, preservatives, antioxidants, wetting agents/surfactants, anti-foaming agents, flocculation modifiers, suspending agents/viscosity-modifiers, flavouring agents, sweetening agents, colorants, humectants, and chelating agents.

As used herein, “cryopreservation” is a process where organelles, cells, tissues, extracellular matrix, organs, or any other biological constructs susceptible to damage caused by unregulated chemical kinetics are preserved by cooling to very low temperatures by freezing (typically −20 to −80° C. using solid carbon dioxide or in some cases to −196° C. using liquid nitrogen). At low enough temperatures, any enzymatic or chemical activity which might cause damage to the biological material in question is effectively stopped. Cryopreservation methods seek to reach low temperatures without causing additional damage caused by the formation of ice crystals during freezing.

As used herein, an “aerosol” is a suspension of liquid and solid particles produced for example by an aerosol generator, such as a small-volume nebulizer (SVN), a pressurized metered-dose inhaler (pMDI), or a dry-powder inhaler (DPI). Aerosol deposition is a process of aerosol particles depositing on absorbing surfaces.

Aerosol devices in clinical use produce heterodisperse (also termed polydisperse) particle sizes, meaning that there is a mix of sizes in the aerosol. Specific sizes and/or size ranges are presented in the Summary of the invention above. Monodisperse aerosols, which consist of a single particle size, are rare. Polydisperse aerosols can be defined by the mass median diameter (MMD). This measure determines the particle size (in μm) above and below which 50% of the mass of the particles is contained. This is the particle size that evenly divides the mass, or amount of the drug in the particle size distribution. This is usually given as the mass median aerodynamic diameter, or MMAD, due to the way sizes are measured. The higher the MMAD, the more particle sizes are of larger diameters.

Dry powder formulations can offer advantages, including greater stability than liquid formulations and potential that preservatives may not be required. Powders tend to stick to the moist surface of the nasal mucosa before being dissolved and cleared. The use of bio-adhesive excipients or agents that slow ciliary action may decrease clearance rates and improve absorption. A number of factors like moisture sensitivity, solubility, particle size, particle shape, and flow characteristics will impact deposition and absorption.

According to some embodiments, the pharmaceutical composition administered via inhalation is administered once daily for at least two days. According to some embodiments, the pharmaceutical composition administered via inhalation is administered once daily for at least three days. According to some embodiments, the pharmaceutical composition administered via inhalation is administered once daily for at least four days. According to some embodiments, the pharmaceutical composition administered via inhalation is administered once daily for at least five days. According to some embodiments, the pharmaceutical composition administered via inhalation is administered once daily for at least six days. According to some embodiments, the pharmaceutical composition administered via inhalation is administered once daily for at least a week. According to some embodiments, the pharmaceutical composition administered via inhalation is administered once daily for at least eight days. According to some embodiments, the pharmaceutical composition administered via inhalation is administered once daily for at least nine days. According to some embodiments, the pharmaceutical composition administered via inhalation is administered once daily for at least ten days. According to some embodiments, the pharmaceutical composition administered via inhalation is administered once daily for ten days.

### Examples of Dry Powder Nasal Sprays:

SBNL Pharma (www.snbl.com) recently reported data on a Phase 1 study with a zolmitriptan powder cyclodextrin formulation (μco™ System) for enhanced absorption, described previously in an in vitro study. The zolmitriptan absorption was rapid, and the relative bioavailability was higher than the marketed tablet and nasal spray. The company has a capsule-based, single-dose powder device (Fit-lizer), suitable for administration of a powder formulation according to the present invention. When inserted into a chamber, the top and bottom of the capsule is cut off by sharp blades. A plastic chamber is compressed by hand, compressed air passes through a one-way valve and the capsule during actuation, and the powder is emitted.

Bespak (www.bespak.com), the principle for Unidose-DP™, is similar to the Fit-lizer device. An air-filled compartment is compressed until a pin ruptures a membrane to release the pressure to emit the plume of powder. Delivery of powder formulations of a model antibody (human IgG) has been tested in a nasal cast model based on human MRI images. Approximately 95% of the dose was delivered to the nasal cavity, but the majority of it was deposited no further than the nasal vestibule with only about 30% deposited into deeper compartments of the nasal cavity.

### Examples of Dry Powder Inhalers:

Astra Zeneca markets budesonide powder delivered with the Turbuhaler multi-dose inhaler device modified for nasal inhalation (Rhinocort Turbuhaler®; www.az.com). It is marketed for allergic rhinitis and nasal polyps in some markets as an alternative to the liquid spray.

The Aptar group (www.aptar.com) offers a simple blister-based powder inhaler. The blister is pierced before use and the device nosepiece placed into one nostril. The subject closes the other nostril with the finger and inhales the powder into the nose. A powder formulation of apomorphine for Parkinson's using this blister-based powder inhaler (BiDose™/Prohaler™) from Pfeiffer/Aptar was in clinical development by Britannia, a UK company recently acquired by Stada Pharmaceutical (www.stada.de). Such administration devices are contemplated for administering the composition as described herein.

Examples of Inhalers with Liquid Formulations:

Inhalation solution and suspension drug products are typically aqueous-based formulations that contain therapeutically active ingredients and can also contain additional excipients. Aqueous-based oral inhalation solutions and suspension must be sterile. Inhalation solutions and suspensions are intended for delivery to the lungs by oral inhalation for local and/or systemic effects and are to be used with a specified nebulizer, for example Pari Boy Pro which can generate flexible and with variable droplet spectrum for the therapy of severe, chronic respiratory diseases. Unit-dose presentation is recommended for these drug products to prevent microbial contamination during use. The container closure system for these drug products consists of the container and closure and can include protective packaging such as foil overwrap.

For example, amikacin liposome inhalation suspension (ALIS; Arikayce®) is a liposomal formulation of the aminoglycoside antibacterial drug amikacin. The ALIS formulation, administered via inhalation following nebulization, is designed to facilitate targeted and localized drug delivery to the lungs while minimizing systemic exposure. Such administration devices are contemplated for administering the composition as described herein.

Examples of Nasal Sprays with Liquid Formulation:

Nasal spray dispensers are typically non-pressurized dispensers that deliver a spray containing a metered dose of the active ingredient. The dose can be metered by the spray pump or could have been pre-metered during manufacture. A nasal spray unit can be designed for unit dosing or can discharge up to several hundred metered sprays of formulation containing the drug substance. Nasal sprays are applied to the nasal cavity for local and/or systemic effects.

The liquid nasal formulations are mainly aqueous solutions, but suspensions and emulsions can also be delivered. Liquid formulations are considered convenient particularly for topical indications where humidification counteracts the dryness and crusting often accompanying chronic nasal diseases.

For example, the dispensers available for example from Nemera (La Verpillière, France) or Aptar Pharma (Illinois, USA) are preferred. For example, Nemera provides Advancia® nasal spray dispensers, which are high-performing pumps with good dose consistency and prime retention, anti-clogging actuators, no metal contacting the formulation and hygienic anti-actuation snap-on overcaps. As a further example, Aptar Pharma's nasal pump technology removes the need for drug manufacturers to add preservatives to nasal spray formulations. The Advanced Preservative Free (APF) systems use a tip-seal and filter technology to prevent contamination of the formulation. A spring-loaded tip seal mechanism is employed with a filter membrane in the ventilation channel.

Metering and spray producing (e.g., orifice, nozzle, jet) pump mechanisms and components are used for reproducible delivery of drug formulation, and these can be constructed of many parts of different design that are precisely controlled in terms of dimensions and composition. Energy is required for dispersion of the formulation as a spray. This is typically accomplished by forcing the formulation through the nasal actuator and its orifice. The formulation and the container closure system (container, closure, pump, and any protective packaging) collectively constitute the drug product. The design of the container closure system affects the dosing performance of the drug product.

In some embodiments, the multi-use dispenser employs a membrane (preferably of silicone) that prevents bacteria or viruses from entering the reservoir or pump device. In some embodiments, the multi-use dispenser employs a metal-free fluid path, thereby preventing the oxidization of the formulation. In some embodiments, the multi-use dispenser employs a spring-loaded tip seal mechanism, thereby preventing microbes from entering the device between spray events.

In one embodiment, the dispenser is configured for multiple individual spray events of 5 to 1000 μL, preferably 5 to 500 μL, more preferably 10 to 300 μL, more preferably 20 to 200 L.

In one embodiment, the dispenser comprises a total volume of composition of 0.1 to 500 mL, preferably 1 to 100 mL, more preferably 2 to 50 mL, such as for example about 5, 10 or 15 mL.

### Examples of Throat Sprays for Liquid Formulations:

Bona, ShenZhen China, is a supplier of medical packaging and complements. The firm offers a line of long-neck sprays that offer excellent dispensing and dosing, designed for throat sprays and other treatments that require throat targeting. The arm of the dispenser swings 360 degrees for consumer ease of use. The sprayers are suited for medicated throat treatments as well as other treatments that local treatment, for example to protect the throat from viral infections. The dispensers are preferably prepared from pharma-grade PP and PE, and the dispensers can be paired with any bottle size. Currently, the sprayers come in a variety of popular sizes (18/410, 18/415, 20/410, 24/410) and can be set to dispense from 220 microliters to 50 microliters.

### Simultaneous and Sequential Administration

Simultaneous administration of one or more active agents shall mean administration of one or more agents at the same time. For example, in one embodiment, AAT can be administrated together other active agent useful for treating a viral infection or medical condition associated with lung inflammation. In other embodiment, AAT can be administrated by inhalation together by other administration route including oral, nasal, parenteral including intravascular, intraperitoneal, or intramuscular administration. In some embodiments, at least one route of administration to be the recombinant AAT according to the present invention is required, preferably inhalation administration. The other routes of administration can alternatively be a composition comprising human plasma derived AAT or any other recombinantly produced AAT. In embodiments, the administration route applied in a simultaneous manner can also be in a sequential manner.

Sequential administration of one or more active agents shall mean administration of the therapeutic agents in a sequential manner. In one embodiment, each therapeutic agent is administered at a different time. In other embodiments, by administration of two or more therapeutic agents wherein at least two of the therapeutic agents are administered in a sequential manner which is a substantially simultaneous manner. Substantially simultaneous administration can be accomplished, for example, by administering to the subject a single dose having a fixed ratio of each therapeutic agent or in multiple, single doses for each of the therapeutic agents. The one or more therapeutic agents as described herein comprises a least the pharmaceutical composition comprising recombinant AAT protein according to the present invention. In an embodiment, the therapeutic agents comprise further pharmaceutical composition comprising human plasma derived AAT and/or other recombinantly produced AAT protein. In a preferred embodiment, the therapeutic agents comprise further pharmaceutical composition comprising Xanthohumol.

As used herein, the term “simultaneously” refers to administration of one or more agents at the same time. For example, in certain embodiments, administration of AAT in combination with other active agents useful for treating a viral infection or medical condition associated with lung inflammation are administered at simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be affected by any appropriate route including, but not limited to, oral routes, intravenous routes, subcutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by inhalation while the other component(s) of the combination may be administered intravenously. The components may be administered in any therapeutically effective sequence.

The decision for simultaneous and sequential administration can be taken depending on the severity of the disease. For example, for the patient with severe symptoms of Covid-19 receiving the inhalation of AAT, a simultaneous and/or sequential intravenous administration of AAT protein can be decided. For example, for the Covid-19 patient receives the inhalation of AAT wherein said AAT droplets comprise Xanthohumol. For example, for the Covid-19 patient receiving the inhalation of AAT wherein said AAT, a separate dose of Xanthohumol can be administrated simultaneously or sequentially.

### FIGURES

The following figures are presented to describe particular embodiments of the invention, without being limiting in scope.

### BRIEF DESCRIPTION OF THE FIGURES

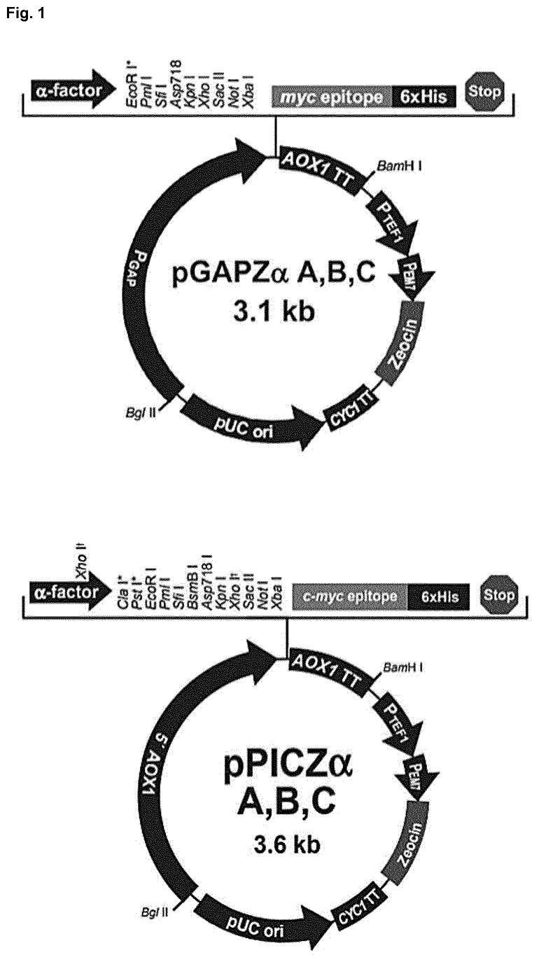

FIG. 1: Vector maps for recombinant protein expression in Pichia pastoris.

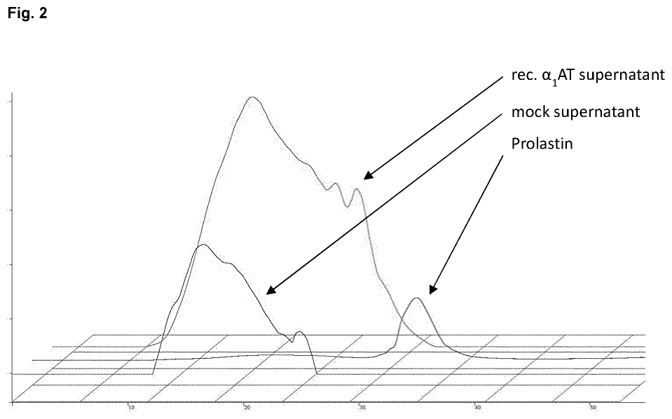

FIG. 2: Purification of supernatants comprising recombinant AAT produced in Pichia pastoris by antion exchange chromatography (AEX).

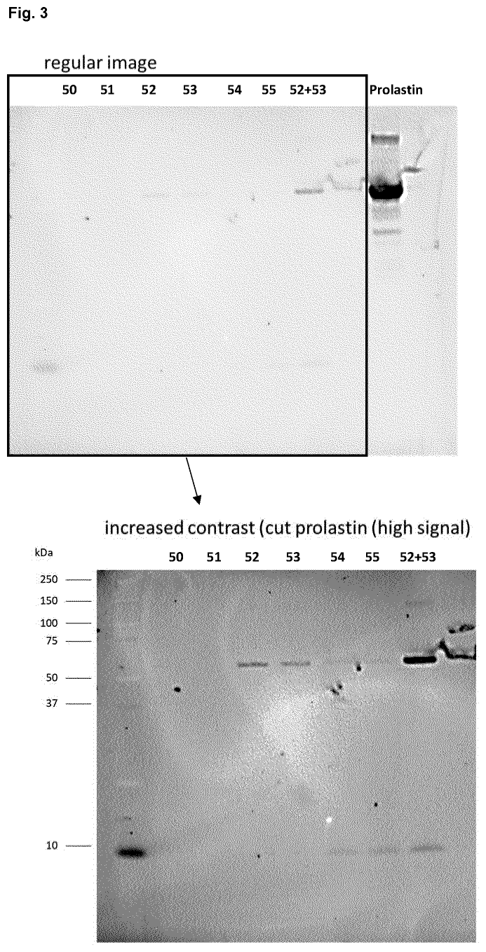

FIG. 3: Western-blot analysis of recombinant AAT fractions obtained from anion exchange chromatography (AEX). The upper and lower panels are of the same image, the lower panel is

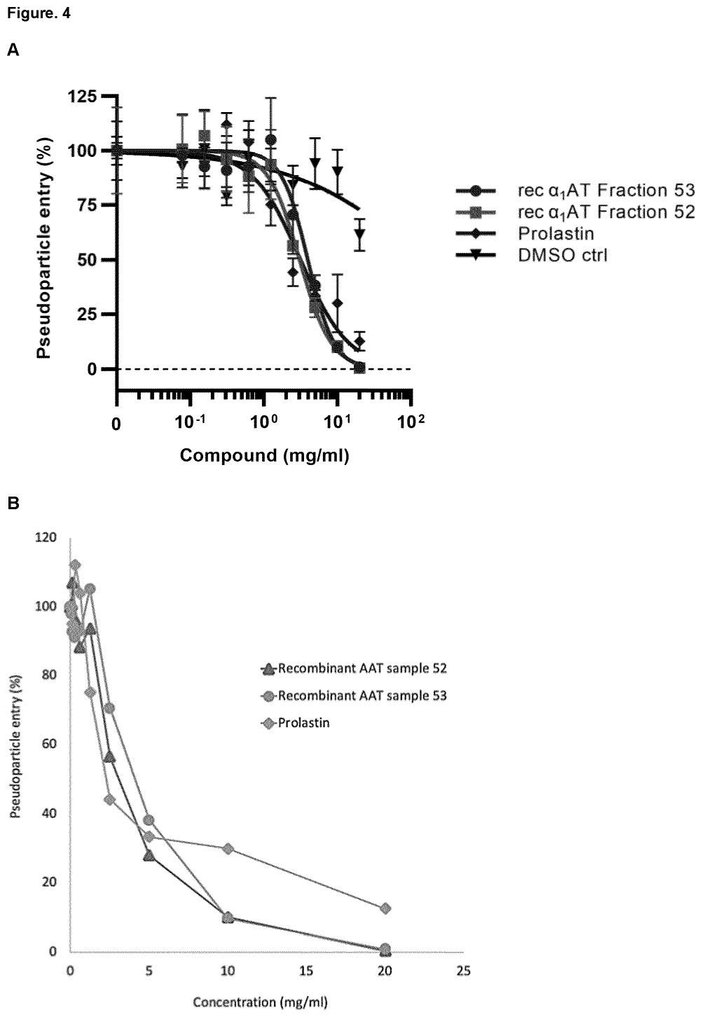

FIG. 4: Test of recombinant AAT for anti-SARS-CoV-2 activity.

### DETAILED DESCRIPTION OF THE FIGURES

FIG. 1: Vector maps for recombinant protein expression in Pichia pastoris. The first vector shown is pGAPZα, comprising a constitutive glyceraldehyde-3-phosphate dehydrogenase (GAP) promoter. The second vector is pPICZα, comprising an inducible alcohol oxidase I (AOX1) promoter.

FIG. 2: Purification of supernatants comprising recombinant AAT produced in Pichia pastoris by anion exchange chromatography (AEX). The human plasma-derived α1AT formulation Prolastin was also applied to SEC. 700 ml of P. pastoris supernatant, from both unmodified WT strains and strains comprising an AAT-expressing vector, were applied to the column. Fractions were obtained in 30s intervals.

FIG. 3: Western-blot analysis of recombinant AAT fractions obtained from anion exchange chromatography (AEX). The upper and lower panels are of the same image, the lower panel is cropped to avoid the strong signal for prolastin. The contrast of the image in the lower panel was increased. 10 μg protein were loaded per lane for each fraction obtained from AEX, 2.5 μg of Prolastin protein was loaded in the control well.

FIG. 4: Test of recombinant AAT for anti-SARS-CoV-2 activity. (A) The experiments were conducted with recombinant AAT fractions 52 and 53 (curve with circle and square), human serum derived AAT Prolastin (curve with rhombus) and DMSO (curve with triangle), respectively. Caco2 (colorectal carcinoma cells, susceptible to SARS-CoV-2) were seeded and on day 1, medium was removed and serum-free medium was added to the cells. Recombinant α1AT samples were solubilized in 10% DMSO in H2O and all compounds and controls were titrated. Compounds were added to the cells, incubated for 1 h at 37° C. The cells were transduced with lentiviral SARS-CoV-2 pseudoparticles. After 48 h, luciferase signals were measured in cell lysates. The read-out provided represents pseudoparticle cell entry (y-axis) against increasing concentration of protein administered to the cells (log; x-axis). (B) The data from the experiment presented in (A) is shown, plotting pseudoparticle cell entry (y-axis) against increasing concentration of protein administered to the cells (linear; x-axis). Fractions 52 (triangle) and 53 (circle) are shown in comparison to Prolastin (diamond).

### EXAMPLES

The following examples are presented to describe particular and potentially preferred embodiments of the invention, indicating practical enablement, without being limiting in scope.

### Example 1: Exemplary Outline for the Preparation of Recombinant AAT

The following general outline for recombinant AAT expression and preparation is provided:

### 1. Fermentation

Fermentation of yeast cultures modified to recombinantly express human AAT is typically carried out in a bioreactor. The yeast cultures are stimulated to produce alpha-1-antitrypsin under optimal conditions in terms of temperature, oxygen concentration, substrate concentration and the like. In embodiments, this process ends at a point in time when the synthesis rate of the yeast cultures decreases. In embodiments, this lasts about 36 to 144 hours, preferably 48 to 120 hours, more preferably 72 to 96 hours.

### 2. Purification

Subsequently, the content of the reactor can be harvested, preferably the culture supernatant is obtained and subsequently processed. The harvested fermentate consists of water, the yeast cultures, residues of the different substrate and auxiliary substances that stabilize the process. The pure alpha-1-antitrypsin must be separated from this mixture. This purification takes place in different steps including separation of yeast culture supernatant and subsequent separation via chromatography columns. At the end of this process, the alpha-1-antitrypsin is available in pure form.

### 3. Preservation

AAT, like all other proteins, is likely subject to a biological decay process without further treatment. This can be stopped by cooling at −80°. However, this procedure is under some conditions not optimal. For this reason, the substance may optionally be freeze-dried (lyophilized) and can thus be stored and transported without any disruption of stability. By adding water, the active substance is returned to its liquid state, for example immediately before each use. This process is state of the art and is used for almost all protein preparations.

### 4. Packing

The “fill and finish” includes the division into the desired individual doses (for example 100 mg) as well as sterile packaging. Depending on the final product, different packaging forms can be considered.

### Examples 2-5

Examples 2-5 show the detailed workflow for generation of human recombinant AAT in Pichia pastoris. The workflow is composed of transformation of a Pichia pastoris host cell line with an AAT expression plasmid, microscale cultivation, microscale re-cultivation and selection of lead clones. A Pichia pastoris expression strain for the recombinant production of human alpha-antitrypsin (rhAAT) was developed.

### Example 2: Design and Receipt of Synthetic AAT Gene and Vector Construction

The AAT encoding gene sequence was designed for expression P. pastoris.

The gene encoding the AAT protein is according to SDEQ ID NO 4 (nucleotide
sequence of the inserted target gene, without flanking regions used for
cloning purposes, with stop codons):
GAGGACCCTCAAGGTGACGCCGCTCAAAAGACTGATACCTCGCACCACGACCAAGACCACCCAA
CTTTTAACAAGATTACTCCTAATCTAGCTGAGTTCGCATTCTCTCTCTACAGACAACTCGCTCATCA
GTCTAACTCTACGAACATTTTCTTCTCCCCAGTGTCCATTGCAACTGCTTTCGCCATGCTTTCTTT
GGGTACTAAGGCTGACACACACGACGAGATTTTAGAAGGATTGAACTTTAATCTTACCGAAATTCC
TGAGGCCCAAATTCACGAAGGTTTTCAGGAGCTGCTTAGAACTCTTAACCAGCCAGATAGCCAGC
TACAACTTACTACTGGAAATGGACTATTCTTGAGTGAAGGTCTGAAGCTAGTTGACAAATTCCTCG
AAGATGTGAAAAAGTTGTACCATTCTGAGGCTTTCACCGTCAACTTCGGTGACACAGAGGAGGCT
AAAAAGCAGATCAACGACTACGTTGAAAAGGGTACGCAAGGTAAAATCGTTGACCTCGTTAAAGA
GCTTGATAGAGACACCGTATTTGCTCTTGTCAATTACATCTTCTTTAAAGGAAAGTGGGAGAGACC
TTTCGAGGTCAAGGACACCGAGGAGGAAGATTTTCATGTAGATCAAGTCACCACTGTTAAGGTTC
CAATGATGAAGCGTCTCGGTATGTTTAACATCCAACACTGTAAAAAGCTATCTTCTTGGGTCCTTC
TGATGAAGTACCTGGGTAACGCTACTGCAATCTTCTTCCTACCAGATGAAGGTAAGCTCCAGCAC
CTAGAGAACGAACTTACCCACGACATCATTACTAAGTTCCTGGAAAACGAAGACCGTAGATCCGC
TTCCCTGCACCTCCCAAAACTGTCTATCACTGGTACCTATGACCTCAAGTCTGTTCTGGGTCAACT
AGGTATAACCAAAGTGTTTTCAAACGGCGCTGACCTCTCCGGTGTGACTGAAGAGGCACCTCTCA
AGCTGTCCAAGGCCGTTCACAAGGCAGTCCTTACCATTGATGAAAAGGGAACTGAAGCTGCCGG
CGCTATGTTCCTAGAGGCCATCCCAATGTCAATTCCTCCCGAGGTTAAGTTCAACAAGCCATTCG
TCTTCCTAATGATTGAACAAAACACAAAGTCTCCTCTGTTCATGGGAAAGGTTGTTAACCCAACCC
AAAAGTAATAG
The protein sequence for AAT is according to SEQ ID NO 5:
EDPQGDAAQKTDTSHHDQDHPTFNKITPNLAEFAFSLYRQLAHQSNSTNIFFSPVSIATAFAMLSLGTK
ADTHDEILEGLNFNLTEIPEAQIHEGFQELLRTLNQPDSQLQLTTGNGLFLSEGLKLVDKFLEDVKKLYH
SEAFTVNFGDTEEAKKQINDYVEKGTQGKIVDLVKELDRDTVFALVNYIFFKGKWERPFEVKDTEEED
FHVDQVTTVKVPMMKRLGMFNIQHCKKLSSWVLLMKYLGNATAIFFLPDEGKLQHLENELTHDIITKFL
ENEDRRSASLHLPKLSITGTYDLKSVLGQLGITKVFSNGADLSGVTEEAPLKLSKAVHKAVLTIDEKGTE
AAGAMFLEAIPMSIPPEVKFNKPFVFLMIEQNTKSPLFMGKVVNPTQK

Upon receipt of the optimized synthetic gene sequence, the target gene was cloned via a restriction enzyme site e.g. Xhol/Xbal into a suitable expression plasmid, here pPICZα was employed.

After receipt of the synthetic gene in the supplier's plasmid, the dried plasmid was solubilized and transformed into E. coli TOP10F′ cells (NEB-5alpha competent E. coli, C2987I, NEB). The E. coli clone carries the supplier's plasmid harboring the synthetic gene. The transformant was re-streaked on agar-plates and plasmids were isolated via standard plasmid preparation procedures.

The synthetic gene from the backbone of the supplier's plasmid was obtained by digesting the plasmid with double restriction enzymes. The digested plasmid was loaded on an agarose gel and separated by gel-electrophoresis. The band for synthetic gene was cut from agarose gel, purified and eluted, which was ready for ligation.

The double digested synthetic gene is ligated into a double digested yeast expression plasmid. The transformant is re-streaked on agar-plates and plasmids were isolated via standard plasmid preparation procedures. The synthetic gene was separated from the backbone of the yeast expression plasmid by double digestion with restriction enzymes. The band size of the digested plasmid was checked by agarose gel-electrophoresis. The selected plasmid was also sequenced using suitable primers.

The positive clones were then re-streaked on agar-plates for larger plasmid preparation (for transformation into Pichia). The prepared plasmid was then desalted over a membrane (MCE membranes, 0.025 μM, Millipore VSWP01300) and the DNA concentration was determined by spectrophotometric measurement and adjusted to approx. 1 μg/μL.

### Example 3: Workflow in Pichia pastoris

All media components used in experiments with P. pastoris during growth phases, transformation, regeneration phases (during transformation) and storage of yeast colonies were certified to be animal-free with respect to direct content, direct contact or possible contamination. The following media was employed:

YPhyD liquid medium (1% yeast extract, 2% phytone, 2% dextrose), YPhyD solid medium (as above, plus 2% w/v agar, antibiotic supplementation as required), BMD liquid (1.34% YNB, 2% dextrose, 0.2M sodium phosphate buffer pH 6.0, 4× 10-5% Biotine).

Electroporation for all transformations was carried out using competent expression cell strain of Pichia pastoris X33, applying a standard procedure and standard equipment for electroporation.

### Example 4: Microscale Cultivation in 96-Deep Well Plates

For screening, single colonies were picked from transformation plates into single wells of 96-deep well plates filled with optimized cultivation media. Corner wells of some plates were inoculated with a mock strain to serve as a matrix for analysis.

After an initial growth phase to generate biomass (BMD solid), expression from the AOX1-promoter was induced by addition of an optimized liquid mixture containing a defined concentration of methanol. At defined points of time, further induction with methanol was performed.

After a total of 72 hours from the initial methanol induction, all deep well plates were centrifuged and supernatants of all wells were harvested into stock microtiter plates for subsequent analysis. After selection of particular strains from screening results, these strains were re-streaked onto non-selective agar-plates in a manner that yields single, isolated colonies per strain. Per strain cultivated in rescreening, six of these individual colonies were inoculated into single wells of 96-deep well plates filled with optimized cultivation media, and treated as described above.

### Example 5: Analysis of Target Protein Expression

Supernatants were assessed for target protein expression based on molecular weight comparisons of samples run on SDS-PAGE gels and western blot. Pre-prepared AAT samples were used as a control.

For SDS-PAGE, diluted or undiluted samples were mixed with 4×LDS sample buffer and 10× Novex reducing agent (both Thermo Scientific) and incubated at 70° C. for 10 min. Samples were then loaded on a Bolt Bis-Tris 4-12% Gel and run with MES buffer. SeeBlue Plus2 Pre-Stained Standard was included as a molecular weight marker (all Thermo Scientific).

For Western Blot, protein transfer from a pre-run gel was performed by dry blotting using the iBlot 2 Gel Transfer Device with dedicated Novex PVDF transfer stacks. The membrane was subsequently saturated with PBS, 0.1% Tween-20, 5% BSA for 30 min at RT with gentle shaking.

For AAT-specific detection the membrane was incubated for 30 min with gentle shaking at RT using the primary AAT-Antibody (SIGMA, SAB4200196-200 μL) diluted 1:750 in PBS, 0.1% Tween-20, 3% BSA. After triple washing step (5 sec, 5 min and 10 min with PBS-0.1% Tween20 under vigorous agitation at ˜200 rpm), the secondary anti-mouseAb-antibody (SIGMA Anti-Mouse IgG-Peroxidase A2554-1 mL) diluted 1:2,000 in PBS, 0.1% Tween-20, 1% BSA was added for 20 min. The membranes were then washed again and color was developed by adding TMB ultra substrate (Thermo Scientific).

SDS-PAGE gels and western blot analysis revealed expression of AAT at a molecular weight comparable to the standard preparation.

Additionally, supernatants were assessed for target protein expression based on microfluidic capillary electrophoretic separation.

A method involving microfluidic capillary electrophoretic separation (GXII, CaliperLS, now Perkin Elmer) and subsequent identification of the target protein based on its size was established. Briefly, several μL of all culture supernatants are fluorescently labeled and analyzed according to protein size, using an electrophoretic system based on microfluidics. Internal standards (contained in supplied solutions from supplier) enable approximate allocations to size in kDa and approximate concentrations of detected signals. Bovine serum albumin (BSA) was used as calibrator for apparent molecular weight and concentration after dilution in mock strain matrix to known concentration.

Procedure: 5 μL sample (from de-glycosylation, see below) is admixed with 8 μL sample buffer (Perkin Elmer, LDS-containing, pH7.58) containing appropriate amount of reducing agent (then pH7.37), and heated for 5 minutes at 95° C. Subsequently, 32 UL of ddH2O is added, and samples are applied to mCE after centrifugation at 4,000 rpm for 3 minutes (to pellet potential aggregates).

Estimated concentrations of rhAAT were determined in supernatants, calculated by comparison of specific peak area with peak area of BSA present in known concentration. Various yeast clones were tested and showed estimated rhAAT amounts of 2 to 11 mg/mL.

### Example 6: Testing Antiprotease Activity of Recombinant AAT Produced in Pichia pastoris

### Compounds:

Prolastin: human serum derived AAT.
Camostat mesylate: trypsin-like serine protease inhibitor
rhAAT: recombinant AAT produced in Pichia pastoris

### Materials & Methods

The respective protease (TMPRSS2 or neutrophil elastase) is mixed with a serial dilution of rhAAT, camostat mesylate or Prolastin resulting in the formation of a complex between the test compound and the protease. Subsequently, a fluorogenic reporter peptide substrate is added. Upon cleavage of the reporter peptide substrate by the protease, a fluorescent dye is set free. Hence, the fluorescence intensity acts as signal for the remaining protease activity.

### TMPRSS2 Activity Assay:

For assessing the activity of recombinant human TMPRSS2, 25 μl of serially diluted Prolastin, camostat mesylate or rhAAT is incubated with 25 μl of 2 μg/ml recombinant TMPRSS2 enzyme (LSBio #LS-G57269) in assay buffer (50 mM Tris-HCL, 0.154 mM NaCl pH 8.0) for 15 min at 37° C. Next, 50 μl of 20 μM BOC-Gln-Ala-Arg-AMC protease substrate (Bachem #4017019) is added and incubated for 2 h at 37° C. Fluorescence intensity is measured after 2 h at an excitation wavelength of 380 nm and emission wavelength of 460 nm in a Synergy™ H1 microplate reader (BioTek) with Gen5 3.04 software. The assay is performed in a 96 well plate with flat bottom.

### Neutrophil Elastase Activity Assay:

Neutrophil Elastase activity is measured by mixing 25 μl of serially diluted Prolastin, camostat mesylate or rhAAT with 25 μl of 2 ng/μl recombinant neutrophil elastase (Merck Millipore #324681) in assay buffer (50 mM Tris, 1 M NaCl, 0.05% (w/v) Brij-35, pH 7.5) for 15 min at 37° C. Next, 50 μl of 200 μM of MEOSUC-Ala-Ala-Pro-Val-AMC substrate (Bachem #4005227) is added and incubated at 37° C. Fluorescence intensity was measured after 5 minutes at an excitation wavelength of 380 nm and emission wavelength of 460 nm in a Synergy™ H1 microplate reader (BioTek) with Gen5 3.04 software. The assay is performed in a 96 well plate with flat bottom.

### Results:

All three test compounds inhibit the activity of TMPRSS2 and neutrophil elastase. IC50 analysis of rhAAT, Prolastin and camostat mesylate is performed. rhAAT inhibits TMPRSS2 proteolytic activity in a dose-dependent manner with an IC50 of ˜350 nM.

### Example 7: Testing Anti-SARS-CoV-2 Activity with Pseudotype Virus Assay

### Introduction

The infection of a target cell by a virus is governed by the viral surface glycoprotein. Viral pseudoparticles are engineered viruses that carry the glycoprotein (gp) of a foreign virus on their surface. For example, the pseudoparticles used in this study are based on an HIV-1 virion that is covered with the spike gp of SARS-CoV-2 or the gp of vesicular stomatitis virus (VSV-G). Hence, they will enter cells by the same mechanism as SARS-CoV-2 or VSV-G, respectively. However, pseudoviruses lack crucial genetic information for their replication, instead they deliver a reporter gene to the infected cell. Therefore, pseudoparticles allow the investigation of highly pathogenic viruses in a safe and high throughput manner.

### Materials & Methods

To test the activity of rhAAT against SARS-CoV-2 pseudoparticles, cells are treated with serial dilutions of rhAAT, Prolastin and camostat mesylate allowing complex formation of rhAAT and TMPRSS2. Next, the cells are inoculated with pseudoparticles carrying the SARS-CoV-2 spike gp or VSV-G, and the viral entry is quantified after 2 days by measuring the reporter gene activity.

### Generation of Lentiviral Pseudoparticles:

For the generation of lentiviral SARS-CoV-2 (LV (Luc)-CoV-2) pseudoparticles, 900,000 HEK293T cells are seeded in DMEM medium supplemented with 10% fetal calf serum, 2 mM L-glutamine, 100 U/ml penicillin and 100 μg/ml streptomycin in a six-well plate. The next day cells are transfected with 0.49 μg of pCMVdR8_91 (encoding a replication-deficient lentivirus), 0.49 μg of pSEW-Luc2 (encoding a luciferase reporter gene, both kindly provided by Prof. Christian Buchholz, Paul-Ehrlich-Institute, Germany), and 0.02 μg of either pCG1-SARS-2-SΔ18 (WT), pcDNA3_1 SARS-CoV-2-Sd19B.1.617.2_4377 (B.1.617.2, Delta) or pCG1_SARS-2-SΔ18 (BA.4 and BA.5, Omicron) by mixing the plasmid DNA with PEI at a 1:3 ratio in serum-free medium.

After 20 min incubation at RT, transfection mix is added to cells dropwise. The cells are washed 8 h post transfection and a growth medium containing 2.5% FCS was added. At 48 h post-transfection, pseudoparticle containing supernatants are harvested and clarified by centrifugation for 5 min at 450 g. Virus stocks are aliquoted and stored at −80° C. until use.

### Pseudoparticle Inhibition Assay:

One day prior to transduction, 10,000 Caco2 cells are seeded in DMEM supplemented with 10% fetal calf serum, 2 mM L-glutamine, 100 U/I penicillin, 100 mg/ml streptomycin, 1× non-essential amino acids and 1 mM sodium pyruvate in a 96-well flat bottom plate. The next day, the medium is replaced by 60 μl serum-free growth medium, and cells are treated with serially diluted rhAAT, Prolastin and camostat mesylate for 1 h at 37° C. followed by transduction of cells with 20 μl of respective lentiviral pseudoparticles. A VSV-G spiked lentiviral pseudoparticle serves as negative control.

Transduction rates are assessed by measuring luciferase activity in cell lysates at 48 h post-transduction with a commercially available kit (Luciferase Assay System, Promega) in an Orion II microplate reader with simplicity 4.2 software. Values for untreated controls are set to 100% transduction.

### Results:

rhAAT inhibits the entry of SARS-CoV-2 similar to Prolastin and camostat mesylate. VSV-G pseudoparticles are not affected by the test compounds and could enter the cells. rhAAT is nontoxic for Caco-2 cells in the tested concentrations. The experiment shows a comparative analysis of pseudoparticle entries in Caco2 cells which are treated with rhAAT, Prolastin and camostat mesylate, respectively. Cells treated with rhAAT at a concentration of 20 mg/ml show almost no pseudoparticle entry.

Examples 6 and 7 are based in part on experimental settings referred to Azouz et al. 2021 (Pathog Immun. 2021 Apr. 26; 6 (1): 55-74).

### Example 8: In Vitro Treatment of the Human Airway Epithelial Cells Infected with SARS-CoV2 with Prolastin and Purified Recombinant AAT Produced in Pichia pastoris

This example is designed to test whether the recombinant AAT produced in Pichia pastoris inhibits SARS-CoV2 replication in human primary airway epithelial cells more effectively than Prolastin. The experiment setting refers to Wettstein et al. 2021 (Nat Commun 12, 1726).

Small airway epithelia cells (SAECs) are treated with Prolastin and recombinant AAT separately after being infected with SARS-CoV-2. Immediately after the inoculum was removed and supernatants were harvested several days post infection and subjected to RT-qPCR specific for SARS-CoV-2.

The human airway epithelial cells (HAEC) are exposed to Prolastin and recombinant AAT separately and then inoculated with SARS-CoV-2. Cells were fixed at day 1, 2 and 3 post infection, stained with DAPI, a SARS-CoV-2 specific spike antibody and α-tubulin-specific antibody.

### Human Small Airway Epithelial Cells:

Human Small Airway Epithelial Cells (Lonza, CC-2547, batch: 18TL082942, donor: 68 years, female) are cultivated in SAGM™ Small Airway Epithelial Cell Growth Medium (Lonza, CC-3118). Alternatively, differentiated air-liquid interface cultures of human airway epithelial cells (HAECs) are generated from primary human basal cells isolated from airway epithelia.

Cells are expanded in a T75 flask (Sarstedt) in Airway Epithelial Cell Basal Medium supplemented with Airway Epithelial Cell Growth Medium SupplementPack (both Promocell). Growth medium is replaced every two days. Upon reaching 90% confluence, HAECs are detached using DetachKIT (Promocell) and seeded into 6.5 mm Transwell filters (Corning Costar). Filters are precoated with Collagen Solution (StemCell Technologies) overnight and irradiated with UV light for 30 min before cell seeding for collagen crosslinking and sterilization. 3.5×10⁴ cells in 200 μl growth medium are added to the apical side of each filter, and an additional 600 μl of growth medium is added basolaterally. The apical medium is replaced after 48 h. After 72-96 h, when cells reached confluence, the apical medium is removed and basolateral medium is switched to differentiation medium. Differentiation medium consisted of a 1:1 mixture of DMEM-H and LHC Basal (Thermo Fisher) supplemented with Airway Epithelial Cell Growth Medium SupplementPack and is replaced every 2 days. Air-lifting (removal of apical medium) defined day 0 of air-liquid interface (ALI) culture, and cells were grown at ALI conditions until experiments are performed at day 25-28. To avoid mucus accumulation on the apical side, HAEC cultures are washed apically with PBS for 30 min every 3 days from day 14 onwards.

### SARS-CoV-2 Infection of HAECs:

Immediately before infection, the apical surface of HAECs grown on Transwell filters are washed three times with 200 μl PBS to remove accumulated mucus. Then, 10 μM of α1AT or 5 μM remdesivir are added into the basal medium and onto the apical surface. Cells are infected with 9.25×10² plaque-forming units (PFU) of SARS-CoV-2 (BetaCoV/France/IDF0372/2020). After incubation for 2 h at 37° C., viral inoculum is removed and cells are washed three times with 200 μl PBS and again cultured at the air-liquid interface. At 1, 2, and 3 days post infection, cells are fixed for 30 min in 4% paraformaldehyde in PBS, permeabilized for 10 min with 0.2% saponin and 10% FCS in PBS, washed twice with PBS and stained with anti-SARS-CoV-2 spike (ab252690, Abcam) and anti-alpha-tubulin (MA1-8007, Thermo Scientific) diluted 1:300 to 1:500, respectively, in PBS, 0.2% saponin and 10% FCS over night at 4° C.

Subsequently, cells are washed twice with PBS and incubated for 1 h at room temperature in PBS, 0.2% saponin and 10% FCS containing AlexaFluor 488-labeled anti-rabbit and AlexaFluor 647-labeled anti-rat secondary antibody, respectively (all 1:500; Thermo Scientific) and DAPI+phalloidin AF 405 (1:5000; Thermo Scientific). Images are taken on an inverted confocal microscope (Leica TCS SP5, Leica Microsystems, Leica application suite version 2.7.3.9723) using a ×40 lens (Leica HC PL APO CS2 40×1.25 OIL). Images for the blue (DAPI), green (AlexaFluor 488) and far-red (AlexaFluor 647) channels are taken in sequential mode using appropriate excitation and emission settings that were kept constant for all the acquisitions. For quantification, randomly chosen locations in each filter are selected and z-stacks were acquired. A maximum z projection was performed and anti-SARS-CoV-2 positive cells per area (0.15 mm2) are visually identified and counted.

### Results:

Recombinant AAT inhibits SARS-CoV-2 replication in primary human airway cells more effectively than Prolastin. Recombinant AAT present during infection reduce viral titer than Prolastin. In infected, recombinant AAT treated HAECs, SARS-CoV-2 spike expression is less detectable than Prolastin treated HAECs.

### Example 9: Curative Trial Using AAT Inhalation

In the context of individual self-studies, patients were treated via inhalation with AAT (Prolastin, Grifols, Spain) after being informed of the protocol. A Pari Boy Pro (Pari, Starnberg, Germany) was used as s nebulizer. Thereby, 100 mg AAT were dissolved in 8 ml saline and added to the container of the nebulizer.

Patient 1, Male, 52 Years Old, with Risk Factor Smoking, had not been Vaccinated.

In the course of the treatment:

On day 1, Patient 1 tested positive for Covid-19 with PCT test and had mild symptoms similar with a flu-like infection.

On day 2, Patient 1 tested negative for Covid-19 with rapid test and had no symptoms. He inhaled 100 mg Prolastin.

From day 3 to day 5, Patient 1 tested negative for Covid-19 with rapid tests and had no symptoms.

Patient 2, Female, 25 Years Old, with Risk Factor Smoking.

Patient 2 had been vaccinated with Comirnaty twice. Second vaccination was received 3 months prior to getting Covid-19.

On day 1, Patient 2 tested positive for Covid-19 twice with a rapid test and had fever, sore throat, fatigue, and a decreased sense of taste. Patient 2 inhaled 100 mg AAT for three times at intervals of 4 hours.

On day 2, the symptoms were significantly improved. Patient 2 inhaled 100 mg AAT on day 2.

On day 3, Patient 2 had no symptoms and inhaled 100 mg AAT. Patient 2 tested negative for Covid-19 with a PCR test.

### Example 10: Recombinant AAT Production from Pichia pastoris

As an alternative to Examples 2-5, the following experimental validation of recombinant AAT produced in Pichia pastoris was carried out.

A codon optimized AAT gene for expression in P. pastoris was designed with GENEius from Eurofins, which was provided in a pEX-A258 vector.

The codon optimized gene encoding the AAT protein is according to
SDEQ ID NO 2:
GAAGATCCACAAGGTGATGCTGCTCAGAAAACAGACACCTCACACCATGATCAAGATCATCCGAC
ATTTAACAAGATCACACCTAACCTTGCAGAGTTCGCCTTTTCCTTGTATCGTCAGCTTGCTCATCA
AAGCAACTCGACGAACATTTTCTTTTCCCCAGTAAGTATTGCAACTGCATTTGCTATGCTATCGTT
GGGTACCAAAGCTGACACTCATGACGAAATATTGGAGGGTCTAAACTTTAACTTGACAGAAATCC
CCGAAGCCCAAATTCATGAGGGATTTCAAGAGTTGTTGAGAACTCTAAACCAACCTGACTCTCAA
CTGCAGTTAACTACCGGTAATGGACTGTTCTTAAGCGAAGGTTTAAAATTGGTCGATAAGTTCCTT
GAGGACGTTAAGAAGTTGTATCACTCTGAGGCTTTTACGGTCAATTTCGGAGATACTGAGGAAGC
CAAGAAACAAATCAATGACTACGTGGAAAAGGGAACTCAAGGCAAGATCGTTGACTTGGTGAAAG
AACTGGATAGAGATACCGTATTTGCTTTAGTGAACTACATCTTCTTTAAAGGGAAATGGGAAAGAC
CATTCGAGGTCAAGGATACTGAGGAGGAAGATTTTCACGTCGACCAGGTAACCACTGTTAAGGTT
CCGATGATGAAACGATTGGGAATGTTCAACATTCAGCACTGTAAGAAGTTGTCAAGTTGGGTTCT
GCTTATGAAGTACTTAGGGAATGCAACTGCCATTTTCTTCTTGCCTGATGAAGGTAAACTGCAACA
TTTGGAAAATGAACTTACACACGATATTATCACCAAATTTCTAGAGAACGAAGATAGGAGATCAGC
CTCTTTGCATTTGCCAAAGCTGTCAATAACAGGTACTTATGACTTGAAATCCGTTCTTGGCCAATT
GGGCATAACTAAGGTGTTTTCTAATGGTGCTGATTTGAGTGGTGTTACAGAGGAAGCTCCCTTAA
AGCTATCTAAGGCTGTTCACAAAGCAGTTCTTACCATTGACGAGAAAGGTACTGAAGCTGCAGGA
GCTATGTTTCTGGAAGCCATTCCTATGTCCATTCCACCTGAAGTTAAATTCAATAAGCCTTTTGTCT
TTCTTATGATTGAGCAGAATACGAAATCTCCATTATTCATGGGAAAGGTAGTGAATCCAACCCAGA
AA
The protein sequence for AAT is according to SEQ ID NO 3:
EDPQGDAAQKTDTSHHDQDHPTFNKITPNLAEFAFSLYRQLAHQSNSTNIFFSPVSIATAFAMLSLGTK
ADTHDEILEGLNFNLTEIPEAQIHEGFQELLRTLNQPDSQLQLTTGNGLFLSEGLKLVDKFLEDVKKLYH
SEAFTVNFGDTEEAKKQINDYVEKGTQGKIVDLVKELDRDTVFALVNYIFFKGKWERPFEVKDATEEE
DFHVDQVTTVKVPMMKRLGMFNIQHCKKLSSWVLLMKYLGNATAIFFLPDEGKLQHLENELTHDIITKF
LENEDRRSASLHLPKLSITGTYDLKSVLGQLGITKVFSNGADLSGVTEEAPLKLSKAVHKAVLTIDEKGT
EAAGAMFLEAIPMSIPPEVKFNKPFVFLMIEQNTKSPLFMGKVVNPTQK

Via restriction digestion and subsequent cloning, the AAT gene was inserted into expression vectors intended for expression in Pichia pastoris. Two suitable expression vectors are shown in FIG. 1. The first vector shown is pGAPZα, comprising a constitutive glyceraldehyde-3-phosphate dehydrogenase (GAP) promoter. The second vector is pPICZα, comprising an inducible alcohol oxidase I (AOX1) promoter.

The construct was cloned into the respective vectors pGAPzα and pPICZα using Gibson assembly. Subsequently, directed mutagenesis was carried out with the Phusion site-directed mutagenesis kit (ThermoFischer), in order to delete the 33 bp linker and His-tag, producing untagged AAT, thus both His-tag AAT fusion proteins and untagged AAT constructs were generated.

Pichia pastoris X33 was subsequently transformed with the AAT expression vectors and cultivated under standard conditions.

Fermentation in a bioreactor with mineral salt medium FM22 (Stratton et al 1998, high cell-density fermentation, Methods Mol Biol.) is carried out, the medium may contain different salts and 40 g/L glycerol as the main carbon source. Defined environmental parameters in the bioreactor are: 30°, pH 6, 1 vvm gassing, 30% dissolved oxygen concentration. All parameters are regulated accordingly in the bioreactor.

To increase cell concentration, glycerol feeding is started as soon as the first batch growth is completed (after approx. 24 h). The increased cell concentration then leads to increased AAT production. If pGAPz is used, the AAT is already produced during growth (constitutive), therefore only harvesting is then required (after 3 days). If pPICz is used, the glycerol feed is followed by another methanol feed to induce the promoter and start AAT synthesis. The process takes correspondingly longer (4 days). In one embodiment, fermentation with pGAPz and glycerol feeding had a cell concentration of 42 g/L (dry weight), a total protein content of 115 mg/L and an AAT concentration of 15 mg/L.

After the fermentation process is completed, the supernatant is first obtained by centrifuging the cells. Optionally, the clear supernatant is then concentrated by ultrafiltration (10 or 30 kDa cut-off), leaving the AAT and other proteins in the retentate. The filtrate can be discarded. The retentate can be purified using any suitable method, for example using metal affinity chromatography, His-Tag purification (if His-tag constructs are employed) or using anion exchange chromatography.

### Example 11: Purification of AAT from Supernatants of Transformed Pichia pastoris X33

As described above, Pichia pastoris X33 was engineered to recombinantly express a1AT into culture supernatants (SN). SN of P. pastoris expressing a1AT (rec. α1AT) and SN of unmodified P. pastoris were purified by anion exchange chromatography (AEX).

In order to gain experience on the elution profile of α1AT and to see if SEC affects the activity of α1AT, the human plasma-derived α1AT formulation Prolastin was also applied to SEC. 700 ml of P. pastoris supernatant were applied to the column, fractions were obtained in 30 s intervals, so that 2 fractions/minute were obtained.

### Results:

The elusion peaks of Prolastin and SN from rec α1AT differ, although the reason for the difference is unclear. Differing glycosylation patterns between human and yeast AAT may play a role in distinct elution profiles. The SN of WT P. pastoris yields a broad peak in AEX. The SN with rec α1AT displays additional peaks absent in the WT SN, indicating the presence of recombinant protein expression. The elution profile is shown in FIG. 2. The additional peaks (˜Fraction 52 and 53) were assumed to contain rec α1AT and were used for further assays.

### Example 12: Western-Blot Analysis of Recombinant AAT Fraction

The recombinant AAT was analysed with Western blot. BCA assay was performed for the samples. The samples were boiled at 70° C. for 10 min and run on 4-12% SDS gel for 90 min at 120V. The samples were blotted on PVDF membrane (semidry) and stained with primary antibody (16382-1-AP, rabbit, 1:1000 diluted) overnight at 4° C. After washing the membrane, the secondary antibody was added and incubated for 30 min at room temperature. After washing, the membranes were imaged.

### Results:

As shown in FIG. 3, recombinant AAT is visible via western blot analysis in fractions 52-57. Of note is that rhAAT provides a clearly defined band, whereas Prolastin shows a smear with multiple side products/impurities, indicating that rhAAT production exhibits improvements in the preparation of AAT.

### Example 13: Testing of Recombinant AAT for Anti-SARS-CoV-2 Activity

### Solubilization of Compounds:

Prolastin (not purified by CEX) was solubilized in H2O. The fractions that putatively contain rec α1AT were solubilized in 10% DMSO.

### Transduction Protocol:

On day 0, 10,000 Caco2 (colorectal carcinoma cells, susceptible for SARS-CoV-2) were seeded. On day 1, medium was removed and serum-free medium was added to the cells. Recombinant α1AT samples were solubilized in 10% DMSO in H2O and all compounds and controls are titrated. Compounds were added to cells, incubated for 1 h at 37° C. The cells were transduced with lentiviral SARS-CoV-2 pseudoparticles. After 48 h, luciferase signals were measured in cell lysates.

For testing, lentiviral pseudoparticles harbouring the SARS-CoV-2 spike protein of Wuhan Hu-1 isolate were used. Pseudoparticles are replication deficient viral particles that display the glycoprotein of another virus. They are used to study viral entry and often carry a reporter gene (here: luciferase) that is expressed upon viral entry into the cell.

TABLE
IC50 values of inhibiting pseudoparticle entry into
Caco2 cells after AAT or control administration:

| Compound | IC50 (mg/ml) |
|---|---|
| rec α₁AT Fraction 53 | 3.96 |
| rec α₁AT Fraction 52 | 3.06 |
| Prolastin | 3.16 |
| DMSO ctrl | n.a |

### Results:

The recombinant α1AT fractions 52 and 53 inhibit the entry of SARS-CoV-2 similar to Prolastin, indicating that the recombinant α1AT is present in these fractions and active. As shown in FIG. 4, a comparative analysis shows pseudoparticle entry in Caco2 cells that were treated with recombinant AAT fractions 52 and 53 (curve with circle and square), human serum derived AAT Prolastin (curve with rhombus) and DMSO (curve with triangle), respectively. At the concentration of 20 mg/ml, the cells treated with recombinant AAT show complete inhibition of pseudoparticle entry whereas still about 10% pseudoparticle entry occurs in the cells treated with Prolastin. FIGS. 4A and B indicate a greater inhibition of pseudoparticle entry by fractions 52 and 53 compared to Prolastin at 10 and 20 mg/mL protein concentrations.

Of note is that the inhibitory curves for Prolastin and rhAAT are of a different shape. An apparently more unspecific inhibition is observed for Prolastin, as derived from the curve shape with an onset of unspecific inhibition at lower concentrations, and not reaching complete inhibition at high doses. A steeper curve shape with a clear inflection point is seen for rhAAT, indicating a more specific inhibition by rhAAT. Prolastin may be less specific, or comprise additional impurities, leading to unspecific inhibitory effects.

As can be derived from the preliminary IC50 values above, fraction 53 with AAT isolated from P. pastoris shows greater activity in the pseudoparticle assay employed, compared to Prolastin. Tests are ongoing using AAT from various P. pastoris expression systems, using controlled AAT concentrations and various purification techniques, and in comparison to Prolastin.

Additional evaluation of rhAAT in comparison to Prolastin is ongoing, whereby IC90 values (also highly relevant for viral inhibition assays) may play a role in assessing rhAAT activity. From initial assessments, IC90 values appear to be different for Prolastin and rhAAT when derived from FIG. 4. Initial findings derived from this assay reveal an estimated IC90 of 20 mg/mL for Prolastin and 8 mg/mL for rhAAT, again indicating an improvement in rhAAT over Prolastin.

1. A method for treating a viral respiratory infection, comprising administering a recombinant alpha1-antitrysin (AAT) protein or a fragment thereof expressed in a genetically modified yeast to a subject in need thereof.

2. The method according to claim 1, wherein the viral respiratory infection is a SARS Coronavirus infection.

3. The method according to claim 2, wherein the viral respiratory infection is SARS-CoV-2.

4. The method according to claim 1, wherein the viral respiratory infection is an influenza virus infection or respiratory syncytial virus (RSV).

5. The method according to claim 1, wherein the AAT protein or fragment thereof comprises a sequence according to SEQ ID NO 3 or 5 or is encoded by SEQ ID NO 1, 2 or 4.

6. The method according to claim 1, wherein the recombinant AAT protein or fragment thereof has a serum half-life and/or activity not less than AAT purified from human plasma.

7. The method according to claim 1, wherein the recombinant AAT protein or fragment thereof is expressed in a yeast of the family Saccharomycesaceae.

8. The method according to claim 1, wherein the recombinant AAT protein or fragment thereof is expressed in Pichia pastoris.

9. The method according to claim 1, wherein the recombinant AAT protein or fragment thereof comprises post-translational modifications.

10. The method according to claim 9, wherein the post-translation modification is one or more of O-glycosylation, N-glycosylation, N-terminal methionine removal, N-acetylation and/or phosphorylation or any combination thereof.

11. The method according to claim 1, wherein the AAT protein or fragment thereof is administered by inhalation.

12. The method according to claim 1, wherein the AAT protein or fragment thereof is administered by inhalation of a nebulized solution.

13. The method according to claim 1, wherein the viral infection is SARS-CoV-2, the AAT protein or fragment thereof is expressed from Pichia pastoris, and the AAT protein or fragment thereof is administered by inhalation of a nebulized solution.

14. The method according to claim 1, wherein the viral infection is an Influenza virus, and the AAT protein or fragment thereof is expressed from Pichia pastoris, and the AAT protein or fragment thereof is administered by inhalation of a nebulized solution.

15. The method according to claim 1, wherein the viral infection is SARS-CoV-2, and the AAT protein or fragment thereof is expressed from Pichia pastoris and isolated using chromatography, and the AAT protein or fragment thereof is administered by inhalation of a nebulized solution.

16. The method according to claim 1, wherein the AAT protein or fragment thereof is produced from genetically modified yeast, in a method comprising the steps of:
a. culturing a genetically modified yeast comprising an exogenous nucleic acid molecule with an AAT-encoding region operably linked to a promoter or promoter/enhancer combination, wherein said yeast is Pichia pastoris,
b. expressing recombinant AAT or fragment thereof in the cultured yeast, wherein the promoter is glyceraldehyde-3-phosphate dehydrogenase (GAP) or alcohol oxidase I (AOX1), and
c. isolating recombinant AAT or fragment thereof from the culture.

17. The method according to claim 1, wherein the AAT protein or fragment is secreted from the yeast into a culture medium, to a concentration of at least 1 mg protein per litre of liquid medium (mg/L).

18. The method according to claim 1, comprising administering a pharmaceutical composition, said composition comprising the recombinant AAT protein or fragment thereof and a pharmaceutically acceptable carrier.

19. The method according to claim 18, wherein the composition is a solution administered by inhalation, a soluble powder administered by inhalation or a dry powder administered by inhalation.

20. The method according to claim 1, wherein the viral respiratory infection is a respiratory syncytial virus (RSV) infection.
